(19) United States
(12) Patent Application Publication
(10) Pub. No.: US 2019/0350236 A1
(43) Pub. Date: Nov. 21, 2019

(54) METHOD OF PROCESSING SHELLFISH AND RESULTING COMPOSITIONS

(71) Applicant: SANFORD LIMITED, Freemans Bay Auckland (NZ)

(72) Inventor: Hong (Sabrina) TIAN, Freemans Bay Auckland (NZ)

(73) Assignee: SANFORD LIMITED, Freemans Bay Auckland (NZ)

(21) Appl. No.: 16/472,031

(22) PCT Filed: Dec. 20, 2017
(86) PCT No.: PCT/NZ2017/050167

§ 371 Date: Jun. 20, 2019

(30) Foreign Application Priority Data
Dec. 20, 2016 (NZ) 727786

(51) Int. Cl. A23L 17/00; A23L 33/18; A22C 29/02; C12P 7/64; C12P 21/06

(52) U.S. Cl. CPC A23L 17/65; A23L 33/18; A61K 35/616; C12P 7/64; C12P 21/06; A22C 29/02

(57) ABSTRACT

A method of processing whole fresh shellfish, including whole live bivalves, and the resulting compositions in liquid and dried form having a high yield of bioactive components with improved bioavailability. The method includes an enzyme treatment step of applying an enzyme formulation having one or more enzymes to the shellfish, and leaving the shellfish in contact with the enzyme formulation until the flesh and other biological material is substantially separated from the shells or exoskeletons of the shellfish, and liquefying the flesh and biological material using of the same enzyme formulation in the same enzyme treatment step, and/or by applying a different enzyme formulation in the same or one or more subsequent enzyme treatment steps. In a preferred method the shellfish starting material is live at the application of the enzyme. The method does not use mechanical processes to reduce the size of the shellfish before application of the enzyme.

### FIELD OF INVENTION

The present invention relates generally to a method of processing shellfish, including molluscs, crustaceans and echinoderms, and to compositions resulting therefrom. It is particularly, but not solely, directed to the processing of molluscs of the class bivalvia.

### BACKGROUND OF INVENTION

Shellfish have long been part of the diet of human populations. Most of the familiar edible shellfish species such as clams, mussels, cockles, oysters, pipi and scallops belong to a group of molluscs known as bivalves. The term bivalve refers to molluscs having two hinged shells (technically called valves), which are connected together by a flexible ligament along the hinge line. Other familiar edible shellfish species include crustaceans such as shrimp, prawns, scampi, crabs, lobster and crayfish, echinoderms such as sea urchins, and other mollusc species such as abalone or paua.

Extensive research has been conducted in relation to the health benefits and bioactive properties of extracts of shellfish and other edible marine species (Sularia et. al., (2015) Marine-Based Nutraceuticals: An Innovative Trend in the Food and Supplement Industries Mar. Drugs (2015) 13, 6336-6351. For example, the unique properties of the New Zealand green-lipped mussel (Perna canaliculus) have been studied for more than 40 years. It was observed that New Zealand coastal Maori populations historically had lower incidences of arthritis than inland Maori populations. This was attributed to the high consumption of green-lipped mussels by the coastal Maori populations thereby suggesting that the green-lipped mussel species had anti-inflammatory activity. Clinical trials have shown that lipid extracts of Perna canaliculus do have anti-inflammatory activity and can be used in the management of arthritis (Halpern (2000) Anti-inflammatory effects of a stabilized lipid extract of Perna canaliculus (Lyprinol); B00r020ien et al. (2008) Systematic review of the nutritional supplement Perna canaliculus (green-lipped mussel) in the treatment of osteoarthritis Q J Med 2008; 101:167-179). Various types of green-lipped mussel lipid extracts have been commercialised for use in the relief of arthritic symptoms.

The New Zealand green-lipped mussel (Perna canaliculus) also contains high levels of Omega-3 fatty acids and they are a rich source of other beneficial bioactive components including vitamins, minerals, taurine, amino acids, polyphenols, carotenoids and active compounds of glucosaminoglycan (GAG or mucopolysaccharide), collagen and glycogen, some of which have been shown to have positive health effects (Grienke et al. (2014) Bioactive compounds from marine mussels and their effects on human health Food Chemistry 142 (2014) 48-60; Coulson et al and Rainsford et al (2015) Novel Natural Products: Therapeutic Effects in Pain, Arthritis and Gastro-intestinal Diseases, Progress in Drug Research 70).

More than 10 million tons of crustaceans are consumed by humans annually. These are either fished or produced by farms, the majority being species of shrimp and prawns. Other species commonly consumed include crabs, lobsters, crayfish and scampi. Krill and copepods are not as widely fished, but may be the animals with the greatest biomass on the planet, and form a vital part of the food chain. Echinoderms such as Evechinus chloroticus, better known as kina (a sea urchin endemic to New Zealand) have been a traditional component of the Māori diet since pre-European times and have been fished commercially in New Zealand since 1986 in small quantities. These marine species may also contain beneficial bioactive components with potential health benefits.

It is very challenging to extract the beneficial bioactive components from shellfish, because it can be difficult to open the shells or exoskeleton to remove or separate the meat and other biological material from inside the shells or exoskeleton, in a manner which preserves the nature and quantity of bioactive components present in the meat and other biological material. Conventional processing methods to remove the material from inside the shells or exoskeleton typically involve mechanical processing of the shellfish, for example, manual or machine shucking, crushing, or grinding to open or break the shells or exoskeletons in order to access the material inside.

For bivalves, some of these methods involve an initial high temperature blanching process to open the shells prior to shucking. However, the use of high temperatures is not ideal since high temperatures can reduce, change, damage, denature or destroy the beneficial bioactive components in the material inside the shells. More recently, a high pressure process (HPP) has been developed to open the shells, and this method can be operated at lower temperatures if specified. This is typically an expensive batch processing operation with the minimal cost for a commercial scale unit being several hundred thousand US dollars. The HPP process only opens the shells, after which the meat still needs to be removed or separated from the shells and then further processed. Accordingly, multiple processing steps and equipment is required to be used in conjunction with the HPP process.

Consequently, the most common commercial processing methods to open the shells or break the exoskeletons and access the material inside the shellfish are manual methods or mechanical crushing methods which do not require the use of heat (thereby avoiding heat damage to the bioactive components). Manual processes are labour intensive, costly and time consuming therefore making commercial production less efficient. Manual or mechanical methods generally result in low yields because not all of the biological material is removed from the shells or exoskeletons, some of it remains and is discarded as waste. Also, the resulting products tend to have different bioactive components, and likely lower levels of some bioactivity, because some of the components are lost or altered during processing and/or soluble components may be discharged with processing liquid.

Furthermore, the digestive tracts of most shellfish species, including bivalves such as mussels, comprise endogenous enzymes which trigger the biological autolysis process. During mechanical processing of shellfish by for example homogenisation such as crushing and grinding these endogenous enzymes are released and cause degradation of biological material which causes changes in compounds and molecular structures which leads to the loss of bioactive components and functional properties. This can also occur when shellfish is stored, even under refrigeration or freezing, and during post-mortem storage. This is one of the reasons why compositions or extracts produced via conventional processing techniques are inconsistent in terms of structure and any bioactivity.

In order to produce compositions or extracts from the meat inside the shells or exoskeletons, once the meat has been extracted, it is necessary to carry out a size reduction process in order to reduce the meat to particulate form so that it can be processed further into various forms such as powders or oils. Mechanical size reduction techniques are commonly used including homogenising, blending, grinding, mincing, milling, pulverising or centrifuging the meat, usually followed by low temperature drying, such as freeze drying. Sometimes the meat is freeze dried before being subjected to mechanical size reduction techniques and then extraction processes.

The disadvantages associated with mechanical opening and size reduction techniques are many. Firstly, production costs can be high due to equipment costs and additional energy costs of processing, and processing can take a long time. Secondly, due to the many processing steps and transfer of biological material from one piece of equipment to another, the biological material is often exposed to air during the process which causes oxidation, deterioration and contamination, creating a poor quality final product. Thirdly, the processes generally yield poorly soluble extracts due to the biological materials not being fully broken down to release their functional components, which limits the options for further processing (for example, some drying methods are not feasible) and limits the use of the extracts in certain applications such as liquid food applications. Fourthly, the processes generally produce low quantitative yields since biological material may be lost during the various processing steps, for example as material is transferred between equipment, and/or the processing method does not fully utilise or break down the raw material so a large proportion of it is wasted. Fifthly, some processes produce products with very low levels of bioactivity because some bioactive components are either changed (for example, by degradation in autolysis as described above) or lost during processing or the processing method does not fully break down or release the bioactive components. For example, some processes involve cooking the meat from inside the shells or exoskeletons prior to or during mechanical size reduction, which may result in loss of liquid and potential bioactive components, and heat damage to some bioactive components and subsequent loss of bioactive properties in the resulting product.

In other processes, such as that described in U.S. Pat. No. 4,801,453, the meat is manually removed from the mussel shell and then placed in a grinding machine and pulverised into small pieces of about ¾ inch, then freeze dried and crushed into a fine powder. An organic acid or an alkaline metal or earth metal salt is added to the extract to stabilize its activity. This process requires a number of steps and different equipment, with manual removal of the meat from inside the shell being very time-consuming and labour intensive. The addition of anti-oxidants during processing has become essential in these types of processing methods in order to retain the bioactivity of the resulting mussel extracts during the slow process.

Enzyme hydrolysis processes have been used to produce shellfish compositions including mussel extracts, however these processes have either been carried out on mussel material that is already in dried or powdered form, therefore the quality of the material has already been compromised as noted above, or the processes are carried out on post-mortem mussel meat (usually frozen and thawed) that has been manually or mechanically from the shells and is homogenised or comminuted prior to any enzyme treatment step. In some processes the meat is not removed from the shells or exoskeletons but the whole dead shellfish are crushed and then all of the crushed material is subjected to enzyme hydrolysis. In all of these prior art processes the original (formerly live) shellfish starting material has been altered significantly, initially by the death of the shellfish (causing alteration of the biological material via post-mortem biological processes including autolysis) and then by processing steps such as freezing, heating, homogenising, crushing, drying which all result in alteration of the biological material and bioactive components in the shellfish, including by autolysis, denaturation, degradation and oxidisation. These steps can eliminate, destroy or change certain classes of compounds present in the original live shellfish material thereby significantly affecting the bioactive properties and bioavailability of the final product. Furthermore, the duration of prior art enzyme treatment processes are long, generally taking a minimum of three hours of hydrolysis, thereby causing further degradation and oxidation of bioactive components. The addition of anti-oxidants is essential in these processes in order to retain the bioactivity of the resulting mussel extracts.

Existing shellfish extracts, including whole mussel powders and lipid extracts are generally poorly soluble in aqueous media (including the digestive tract) because of their structure and hydrophobic properties. They therefore present considerable formulation challenges and often suffer from poor or irregular bioavailability when given orally or via other routes requiring transmembrane absorption.

### OBJECT OF THE INVENTION

It is an object of the invention to provide an improved method of processing shellfish that ameliorates some of the disadvantages and limitations of the known art, or at least provides the public with a useful choice. It is a further object of the invention to provide increased quantitative and/or qualitative yields of shellfish compositions and/or extracts produced by the method of the invention, or at least to provide the public with a useful choice. It is a further object of the invention to provide improved shellfish compositions and/or extracts, or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY OF INVENTION

According to one aspect of the invention there is provided a method of preparing a liquid composition from whole fresh shellfish starting material, wherein the method comprises at least one enzyme treatment step comprising:
(a) applying an enzyme formulation comprising one or more enzymes to the whole fresh shellfish, and
(b) leaving the shellfish in contact with the said enzyme formulation until the flesh and other target substrates (as herein defined) are substantially separated from the shells or exoskeletons of the shellfish, and
(c) liquefying the flesh and other target substrates by the use of the same enzyme formulation in the same enzyme treatment step, and/or by applying a different enzyme formulation in the same or one or more subsequent enzyme treatment steps.

Preferably the enzyme formulation in step (a) includes at least one proteolytic enzyme.

The method of the invention does not use any mechanical processes to reduce the size of the shellfish starting material before the enzyme treatment step.

Preferably the shellfish starting material is whole live shellfish at or up to the point where the enzyme formulation is applied to the shellfish.

Preferably the method further comprises at least one separation step to remove any solid material and/or non-target substrates from the resultant liquid composition.

Preferably the or each enzyme formulation comprises one or more enzymes suitable for acting on one or more target substrates of the shellfish starting material.

The target substrates can include any biological material present on or in the shells or exoskeletons of the shellfish, including the meat or flesh inside the shells or exoskeletons, chitosan present on the shells or exoskeletons, layers of biological material that might be present inside the shells or exoskeletons (for example, the nacre, prismatic and periostracum layers present in mussels (resembling skin)), ligaments, abductor muscles, teeth, byssus threads (or beards), gut and feet.

Preferably the resultant liquid composition is a stable emulsion-like composition having at least one hydrophobic phase and at least one hydrophilic phase. Preferably the composition comprises a mixture of micro-particles and/or micro-droplets and/or nano-particles and/or nano-droplets. Preferably at least some of the droplets or globules in the hydrophobic phase have a layer surrounding or encapsulating the droplets or globules wherein one or more lipid or lipophilic bioactive components are located inside the droplets or globules and are protected. Preferably the hydrophilic phase comprises one or more bioactive components dispersed or suspended therein.

Preferably the live shellfish is selected from the following species: New Zealand green-lipped mussel (Perna canaliculus), the Asian green mussel (Perna viridis), the Mediterranean blue mussel (Mytilus galloprovincialis), the common blue mussel (Mytilus edulus), California mussel (Mytilus californianus), the brown mussel (Perna perna), other Perna mussel species, the Korean mussel (Mytilus coruscus), the Chilean mussel (Mytilus chilensis), the bay mussel (Mytilus trossulus), the ribbed mussel (Geukensia demissa), the date mussel (Lithophaga lithophaga), and the fresh water Zebra Mussel (Dreissena polymorpha); Brachidontes rodriguezii; Perumytilus purpuratus; Aulacomya ater; Choromytilus chorus; and Modiolus mussel species; all species of clams; all species of cockles; all species of oysters including rock oysters (Saccostrea glomerata), bluff oysters (Ostrea chilensis) and pacific oysters (Crassostrea gigas); all species of pipi (Phaphies species) including toheroa and tuatua; all species of scallops including Golden Bay scallops (Pecten novaezelandiae) queen scallops (Zygochlamys delicatula); all species of cockles including Austrovenus stutchburyi scampi (Metanephrops challengeri), crabs, lobster, crayfish, prawns, krill paua (Haliotis species) and sea urchins including Evechinus chloroticus.

Preferably the shellfish is a species of bivalve, and whole live bivalves are used as the starting material in the process of the invention. Preferably the whole live bivalves are gapped or opened in some manner before or during application of the enzyme formulation. Preferably the bivalves are alive at or up to the point of the gapping or opening step.

Preferably the method of the invention includes at least one warming step carried out before and/or during the enzyme treatment step to condition the shellfish for the enzyme treatment step and/or to activate the enzyme formulation.

When processing whole live bivalves, the warming step is preferably used to open or gap the bivalves prior to, during or after exposure to the enzyme formulation. Alternatively, an HPP process could be used to open or gap the bivalves. Other processes could also be used to open or gap the bivalves such as application of laser means or localized means to the abductor muscles, or creation of small openings in the shell of the bivalves by piercing or lightly cracking the shells. While gentle, non-mechanical methods are preferred, any method could be used that achieves the purpose of exposing at least a portion of the interior of the shells of the bivalves to the enzyme formulation.

Preferably the warming step is carried out by way of application of steam (e.g. flash steam injection or infusion) to achieve and maintain an optimum processing temperature. Preferably the optimum processing temperature is no more than 60° C., preferably in the range of about 20-60° C., and more preferably in the range of about 35-60° C.

Alternatively, the warming step could be carried out by the use of a thermal jacket or other thermal means to warm the shellfish and maintain the shellfish at the optimum processing temperature.

The enzyme formulation(s) can comprise one or more types of enzymes sourced from animal, plant or microbial origins, or a combination of one or more enzymes with one or more acids or alkalis.

Preferably the or each enzyme formulation comprises one or more enzymes selected from the group comprising proteolytic enzymes, lipase, laminarinase, phospholipase, phosphatase, glycogen phosphorylase, glucosyltransferase, glucosidase, proteinase, collagenase, glycogen debranching enzymes, phosphoglucomutase, cellulases, chitinases, polysccharidases, disaccharidases, alginase, amylase, maltase, peptidase, pepsin, thrombin, trypsin, α-Amylase (from malted cereals), ß-Amylase (from sweet potato or malted cereals), actinidin (from kiwifruit), ficin (from figs), bromelain (from pineapple), papain (from Papaya), and enzymes derived from the following microorganisms: Bacillus amyloliquefaciens, Bacillus subtilis, Bacillus licheniformis, Bacillus coagulans, Bacillus acidopullulyticus, Bacillus halodurans, Aspergillus melleus, Aspergillus oryzae, Aspergillus niger, Lactococcus lactis, Geobacillus stearothermophilus, Rhizomucor miehei, Micrococcus luteus, Penicillium funiculosum, Trichoderma reesei, Trichoderma viride, Escherichia coli, Kluyveromyces lactis, Paenibacillus macerans, Chaetomium gracile, Penicillium lilacinum, Saccharomyces cerevisiae, Bacillus circulans, Kluyveromyces marxianus, Trichoderma harzianum, Disporotrichum dimorphosporum, Humicola insolens, Talaromyces emersonii, Rhizopus delemar, Rhizopus oryzae, Rhizopus niveus, Hansenula polymorpha, Penicillium camembertii, Candida rugosa, Mucor javanicus, Penicillium roquefortii, Rhizopus arrhizus, Cryphonectria parasitica, Streptomyces violaceoruber, Klebsiella pneumoniae, Streptomyces mobaraensis, Lactobacillus fermentum, Actinoplanes missouriensis, Microbacterium arborescens, Streptomyces olivaceus, Streptomyces olivochromogenes, Streptomyces murinus, Streptomyces rubiginosus and Clostridium histolyticum.

The enzyme formulation(s) may further include one or more acids or alkalis selected from the group comprising phosphoric acid, sulphuric acid, tannic acid, citric acid, tartaric acid, sodium hydroxide, ammonium hydroxide, magnesium hydroxide and potassium hydroxide.

Preferably a combination of enzymes is used in the or each enzyme formulation, each of which or the combination of which is suitable for acting on one or more target substrates present in the shellfish starting material.

Preferably if a different enzyme formulation is used in the first or a subsequent enzyme treatment step, the different enzyme formulation comprises enzymes suitable for further hydrolysing or liquefying one or more non-protein target substrates.

Preferably the or each enzyme formulation comprises at least one enzyme suitable for acting on myofibrillar protein and/or carbohydrate substrates. Preferably the enzyme(s) is/are selected from the group comprising enzymes derived from bacterial strains that produce subtilisin, including Bacillus amyloliquefaciens, and other similarly acting plant and animal derived enzyme(s) such as amylase and trypsin.

Preferably the or each enzyme formulation comprises at least one enzyme suitable for acting on collagen protein substrates. Preferably the enzyme(s) is/are selected from the group comprising enzymes derived from Bacillus licheniformis, Bacillus subtilis, and Aspergillus niger.

Preferably the or each enzyme formulation comprises at least one enzyme suitable for acting on both myofibrillar and collagen protein substrates. Preferably the enzyme(s) is/are selected from the group comprising cysteine proteases.

Preferably the or each enzyme formulation comprises at least one enzyme suitable for acting on lipid substrates. Preferably the enzyme(s) is/are selected from the group comprising Aspergillus oryzae, carbohydrases, sucrase, amylase, lipase, phospholipase, phosphatase, proteases, esterases, and catalase.

In one preferred embodiment of the invention the or each enzyme formulation comprises a combination of at least two enzymes selected from the group comprising enzymes derived from Bacillus amyloliquefaciens, enzymes derived from Bacillus licheniformis, cysteine proteases, and enzymes derived from Aspergillus oryzae.

Preferably the amount of the or each enzyme included in the or each enzyme formulation is in the range of 0.1-10% calculated based on an estimated amount of the or each target substrate to be treated in the or each enzyme treatment step.

Preferably, no water is added during processing, either before, during or after the enzyme treatment step.

Preferably only one enzyme treatment step is required in the process of the invention. Alternatively, one or more enzyme treatment steps may be carried out consecutively to progressively treat a substantially full range of target substrates present in the shellfish starting material.

Preferably the duration of each enzyme treatment step is less than 120 minutes, more preferably less than 90 minutes and even more preferably is in the range of 15-40 minutes.

A key advantage of the invention is that only a very short enzyme treatment step is required to rapidly separate the target substrates or biological material from the shells or exoskeletons of the shellfish and substantially liquefy the material into the form of an emulsion-like liquid composition. The short enzyme treatment step helps to avoid degradation and oxidation of bioactive components in the shellfish starting material.

Preferably the process further comprises an agitation step carried out during the or each enzyme treatment step, which enables the shellfish to be continually moved and therefore more evenly exposed to the enzyme formulation and the increased temperature if a warming step is employed.

Preferably a separation step is carried out by the use of screens, filters or sieves, or a combination thereof. A series of separation and/or subsequent filtration steps may be used to obtain a liquid composition with a desired particle size or particular food matrix or emulsion-like structure, or for better recovery of certain bioactive components.

Preferably the material remaining after the separation or filtration step(s) is recycled and retreated with one or more different enzyme formulations in one or more subsequent enzyme treatment steps so that the larger biological structures are further liquefied and converted into smaller particles until a composition with the desired particle size or particular food matrix or emulsion-like structure or certain level of bioactive components is achieved in respect of substantially all of the target substrates in the shellfish starting material.

In a preferred embodiment of the invention, the main steps of the process are carried out in a single treatment vessel or chamber. Preferably the treatment chamber is a cylindrical shaped vessel which can be sealed and pressurised.

Preferably the treatment chamber is orientated horizontally or in a sloped position, not vertically.

Preferably the treatment chamber includes at least a sealable opening, a heating means, a dosing system for the enzyme formulation, and an agitating means.

Preferably the dosing system includes an automatic dispensing device connected to a dosing means located inside the treatment chamber.

Preferably the agitating means comprises means which are able to continuously or semi-continuously rotate or agitate the treatment chamber in a wide variety of angles or positions to achieve an even and maximum distribution of heat and enzyme formulation onto and around the shellfish.

Preferably the treatment chamber includes an exhaust system which is activated at the conclusion of the enzyme treatment step to expel the heat or steam and pressure within the treatment chamber.

Preferably one or more filtration steps may be carried out after the separation step to progressively reduce the particle size of the resultant liquid composition. Preferably the liquid composition is filtered to a particle size of less than 200 μm in one filtration step after the separation step.

Preferably the treatment chamber includes a recycling system where the material remaining after the separation step and/or the filtration step(s) (if carried out) can be re-circulated back to the treatment chamber for one or more subsequent enzyme treatment steps.

The resultant liquid composition is preferably stabilised before or after the separation and/or filtration step(s) (if carried out), in order to deactivate the enzyme(s) and to pasteurise or sterilise the liquid composition. Preferably the stabilisation step is carried out by application of heat by a further steam injection or infusion or by means of a heat exchanger to quickly increase the temperature of the composition to above 80° C. for a short time period. Alternatively the stabilisation step is carried out by methods not involving heat treatment, for example, pH or microfiltration or ultrafiltration methods.

In a preferred embodiment the resulting liquid composition is dried.

According to a further aspect of the invention there is provided a method of preparing a dried composition from whole fresh shellfish starting material, wherein the method comprises processing the shellfish starting material in the manner described herein to produce a liquid composition, followed by a drying step.

Preferably drying is carried out by low temperature drying means such as freeze drying, or by flash drying means such as spray drying, fluidized bed drying, vacuum drying or belt drying.

After drying, the dried composition may be ground or milled into a powder.

Preferably the dried composition is further processed into capsule or tablet form with suitable additives and/or excipients.

The compositions of the invention have an increased yield of bioactive components, which are expected to have increased bioavailability due to the unique food matrices or emulsion-like structures produced by the processing method. Advantageously, no antioxidants are required to be added during or after processing in order to maintain the bioactivity of the compositions.

In a further aspect of the invention there is provided a method of preparing a liquid composition from whole fresh shellfish of the species bivalvia, said method comprising the steps of:

gapping or at least partially opening the shells of the bivalves or otherwise exposing at least some of the material inside the shells of the bivalves;

applying an enzyme formulation comprising one or more proteolytic enzymes to the whole fresh bivalves and leaving the bivalves in contact with the said enzyme formulation for a sufficient period of time to substantially separate the target biological material from the shells of the bivalves, and substantially liquefy the target biological material.

Preferably the target biological material is liquefied by the use of the same enzyme formulation in the same enzyme treatment step. Alternatively or additionally, one or more different enzyme formulations could be used in the same enzyme treatment step and/or in one or more subsequent enzyme treatment steps.

Preferably the method comprises a further step of separating the shells and any other non-target biological material from the liquefied composition.

Preferably the bivalves are alive at or up to the point of the gapping or opening step.

Preferably the enzyme formulation comprises one or more enzymes suitable for acting on one or more target substrates of the whole fresh bivalve starting material.

Preferably the bivalves are mussels. More preferably they are green-lipped mussels.

Preferably the liquid composition is a stable emulsion-like composition. Preferably the composition comprises a mixture of micro-particles and/or micro-droplets and/or nano-particles and/or nano-droplets. Preferably at least some of the droplets or globules in the hydrophobic phase have a layer encapsulating the droplets or globules wherein one or more lipid or lipophilic bioactive components are located inside the droplets or globules and are protected. Preferably the hydrophilic phase comprises one or more bioactive components dispersed or suspended therein.

Preferably the bivalves are gapped or opened by a gentle warming step. Alternatively an HPP process could be used to open or gap the bivalves. Other processes could also be used to open or gap the bivalves such as application of laser means or localized means to the abductor muscles, or the shells could be penetrated by piercing or lightly cracking the shells to create openings with minimal disturbance to the biological material inside the shells. While gentle, non-mechanical methods are preferred, any method could be used that achieves the purpose of exposing at least a portion of the interior of the shells of the bivalves to the enzyme formulation.

Preferably the warming step is carried out by way of application of steam (e.g. flash steam injection or infusion) to achieve and maintain an optimum processing temperature.

Preferably the optimum processing temperature is no more than 60° C., preferably in the range of about 20-60° C., and more preferably in the range of about 35-60° C.

Alternatively the warming step could be carried out by the use of a thermal jacket or other thermal means to warm the bivalves and maintain the bivalves at the optimum processing temperature.

Preferably the warming step and enzyme treatment step(s) are carried out in a single treatment vessel or chamber. Preferably the treatment chamber is a cylindrical shaped vessel which can be sealed and pressurised. Preferably the treatment chamber has the features as described above.

Preferably the treatment chamber is orientated horizontally or in a sloped position, not vertically. Advantageously no water is added to the treatment chamber during processing.

The enzyme formulation can comprise one or more types of enzymes sourced from animal, plant or microbial origins, or a combination of one or more enzymes with one or more acids or alkalis, as described more fully above.

For processing bivalves, preferably one of the target substrates is the abductor muscles of the bivalves and at least one enzyme in the enzyme formulation is a proteolytic enzyme suitable for acting on this target substrate in order to facilitate the opening of the shells. Other types of enzymes can then be applied separately or together at the same time or subsequently to act on other target substrates including the flesh and other biological material inside the shells including non-protein substrates.

In a preferred embodiment, for processing bivalves, the enzyme formulation comprises at least one enzyme selected from the group comprising enzymes derived from bacterial strains that produce subtilisin, including Bacillus amyloliquefaciens, and other similarly acting plant and animal derived enzyme(s) such as amylase and trypsin, enzymes derived from Bacillus licheniformis, Bacillus subtilis, and Aspergillus niger, cysteine proteases, enzymes derived from Aspergillus oryzae, carbohydrases, sucrase, amylase, lipase, phospholipase, phosphatase, proteases, esterases, and catalase.

In one preferred embodiment the enzyme formulation comprises a combination of at least two enzymes selected from the group comprising enzymes derived from Bacillus amyloliquefaciens, enzymes derived from Bacillus licheniformis, cysteine proteases, and enzymes derived from Aspergillus oryzae.

Preferably the amount of the or each enzyme included in the or each enzyme formulation is in the range of 0.1-10% calculated based on an estimated amount of the or each target substrate to be treated in the or each enzyme treatment step.

Preferably one or more enzyme treatment steps are carried out consecutively to progressively liquefy a substantially full range of target substrates present in the whole fresh bivalve starting material.

Preferably the specified time period of the or each enzyme treatment step is less than 120 minutes, more preferably less than 90 minutes and even more preferably is in the range of 15-40 minutes.

Preferably one or more filtration steps may be carried out after the separation step to progressively reduce the particle size of the liquid composition. Preferably the liquid composition is filtered to a particle size of less than 200 μm in one filtration step after the separation step.

The liquid composition is preferably stabilised before or after the separation or filtration step(s) (if carried out), in order to deactivate the enzyme(s) and to pasteurise or sterilise the liquid composition.

Preferably the method includes a further step of drying the liquid composition to produce a dried composition.

Preferably the dried composition has high solubility in aqueous media and can be rehydrated to form a stable emulsion-like composition.

In a further aspect of the invention there is provided a liquid or dried shellfish composition produced by one of the methods described herein.

Preferably the liquid and/or dried shellfish composition comprises a high yield of bioactive components or concentrated active ingredients.

Preferably the liquid shellfish composition comprises particles with a mean particle size distribution in the range of 0.1-100 μm, including some nano-particles and/or nano-droplets with sizes in the range of 1-100 nm and some micro-particles and/or micro-droplets with sizes in the range of 100 nm-1 μm. More preferably the majority of the particles and/or droplets in the liquid composition comprise micro-particles and/or micro-droplets with sizes in the range of 100-50,000 nm, and even more preferably in the range of 100-10,000 nm.

Preferably the liquid and/or dried shellfish composition has the properties and/or characteristics of a self-emulsifying composition. Preferably the dried composition when combined with a sufficient amount of water, produces a stable emulsion-like composition.

Preferably the liquid or dried shellfish composition is free of added antioxidants.

The liquid or dried shellfish compositions can be fractionated or extracted to separate the compositions into a lipid or lipid-rich fraction and a hydrophilic or aqueous fraction.

The liquid or dried shellfish compositions or fractions or extracts thereof can be formulated into a wide variety of products including but not limited to, food products, nutraceutical products, pharmaceutical products, veterinary products or cosmetics.

In a further aspect of the invention there is provided a lipid, lipid-rich or hydrophobic shellfish extract, wherein said extract is produced by preparing a liquid or dried shellfish composition by the method described herein or obtaining a liquid or dried shellfish composition made by the method described herein, and extracting and recovering the lipid-rich or hydrophobic fraction from the liquid or dried shellfish composition.

In a further aspect of the invention there is provided an aqueous or hydrophilic shellfish extract, wherein said extract is produced by preparing a liquid or dried shellfish composition by the method described herein, or obtaining a liquid or dried shellfish composition made by the method described herein, and extracting and recovering the aqueous or hydrophilic fraction from the liquid or dried shellfish composition.

This invention may also broadly be said to consist in the parts, elements and features referred to or indicated in the specification of the application, individually or collectively, and any or all combinations of any two or more of the parts, elements or features, and where specific integers are mentioned herein which have known equivalents such equivalents are deemed to be incorporated herein as if individually set forth.

### Definitions

In this specification, unless the context otherwise requires, the following terms shall have the following definitions:

“whole” as used herein in connection with shellfish starting material means shellfish including their shells or exoskeletons that are substantially whole and intact, and substantially unprocessed.

“fresh” as used herein in connection with shellfish starting material means shellfish that is alive or has died less than 12 hours before processing commences, but preferably less than three hours before processing commences.

“bioactive components or bioactive compounds” means any one or more chemical molecules, elements or compounds that has an effect on a living organism, tissue or cell or gene, and includes any molecule(s), element(s) or compound(s) or combinations thereof that are or may be beneficial to the health or wellbeing of humans and other animals.

“food matrices” means the nutritional and structured materials (including nutrient and non-nutrient components) of the compositions of the invention and their molecular relationships (i.e. chemical bonds) to each other.

“emulsion-like composition” in the context of the invention means a liquid composition resembling an emulsion or a colloid, comprising at least one hydrophilic phase or continuous phase and at least one hydrophobic phase or dispersed phase, and which may also comprise some solid particles in solution or suspension. The term includes colloidal suspensions, colloidal emulsions and colloidal dispersions.

“emulsion” includes all types of emulsions, including macro-emulsions, single emulsions, double emulsions, multiple emulsions, micro-emulsions, nano-emulsions, colloidal emulsions and emulsified suspensions.

“enzyme formulation” means a formulation comprising at least one enzyme and includes formulations comprising a mixture of one or more enzymes, and formulations comprising one or more enzymes and one or more other non-enzyme substances.

“opening” or “gapping” as used herein is intended to include any method that achieves the purpose of exposing at least a portion of the interior of the shells of bivalves to an enzyme formulation.

“self-emulsifying” as used herein refers to compositions which spontaneously or with only minimal agitation form a stable emulsion or dispersion when added to an aqueous medium.

“stable” as used herein in connection with the emulsion-like compositions of the invention refers to liquid compositions or rehydrated dried compositions which exhibit no phase separation when kept, without agitation, at room temperature for one hour or longer.

“target biological material” or “target substrates” as used herein refers to any desired biological material present on or in the shells or exoskeletons of the shellfish, namely the meat or flesh inside the shells or exoskeletons, but also including other material such as chitosan present on the shells or exoskeletons, layers of biological material that might be present inside the shells or exoskeletons (for example, the nacre, prismatic and periostracum layers present in mussels (resembling skin)), ligaments, abductor muscles, teeth, byssus threads (or beards), gut and feet.

### DESCRIPTION

The invention will now be described, by way of example only, with reference to the accompanying drawings:

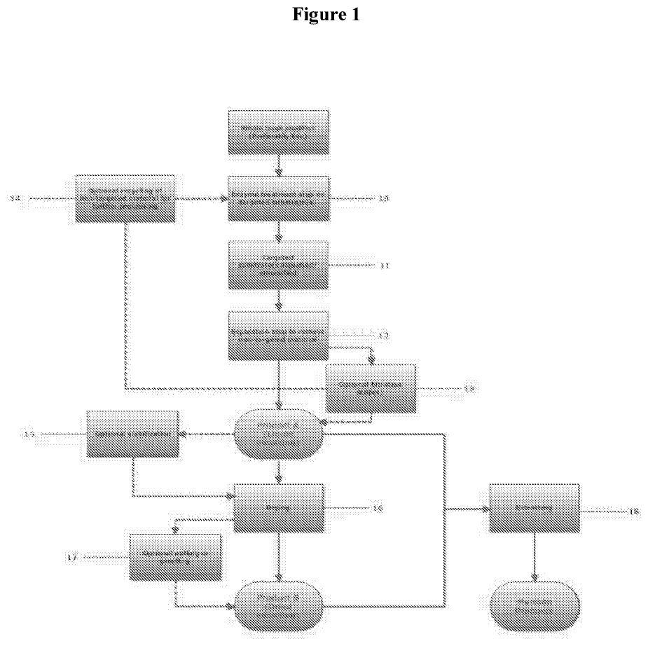

FIG. 1 is a flow chart of a process for producing a liquid or dried composition from shellfish in accordance with a preferred embodiment of the invention.

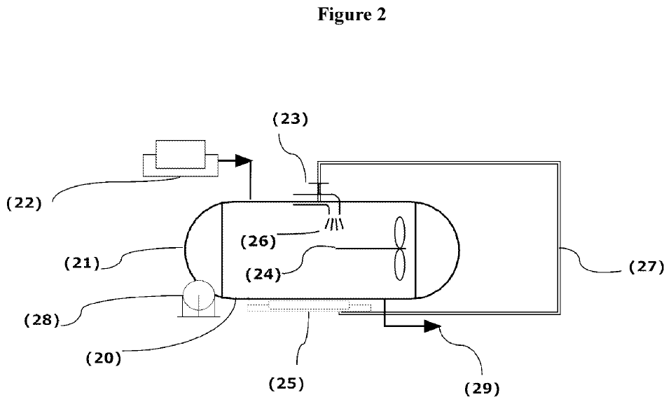

FIG. 2 is a schematic representation of a treatment chamber that may be used in the process of the invention.

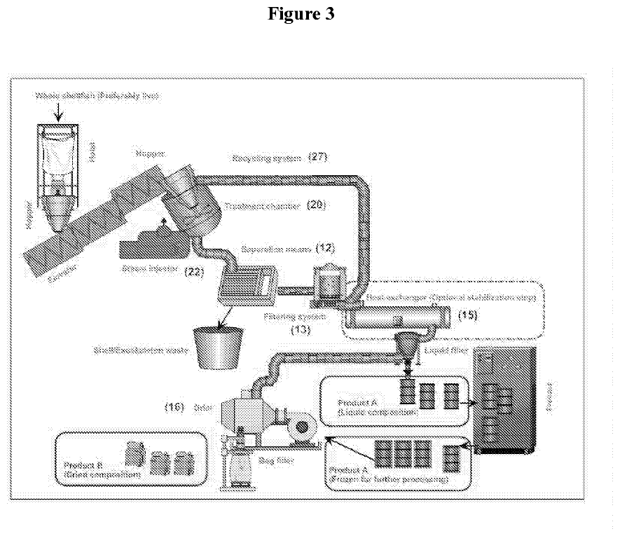

FIG. 3 is a schematic representation of an example of the process of the invention incorporating the treatment chamber of FIG. 2.

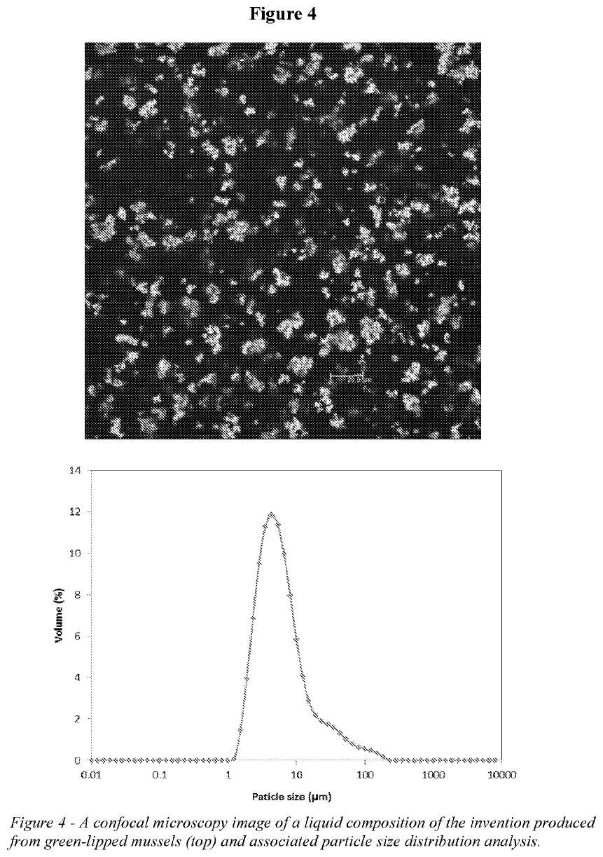

FIG. 4 is a confocal microscopy image and associated graph showing the particle size distribution of a liquid composition of the invention produced from whole live green-lipped mussels.

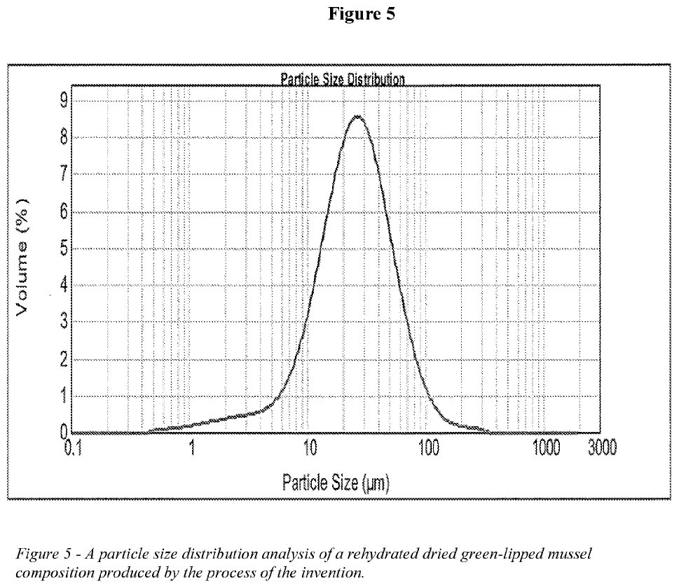

FIG. 5 is a graph showing the particle size distribution of a rehydrated dried composition of the invention produced from whole live green-lipped mussels.

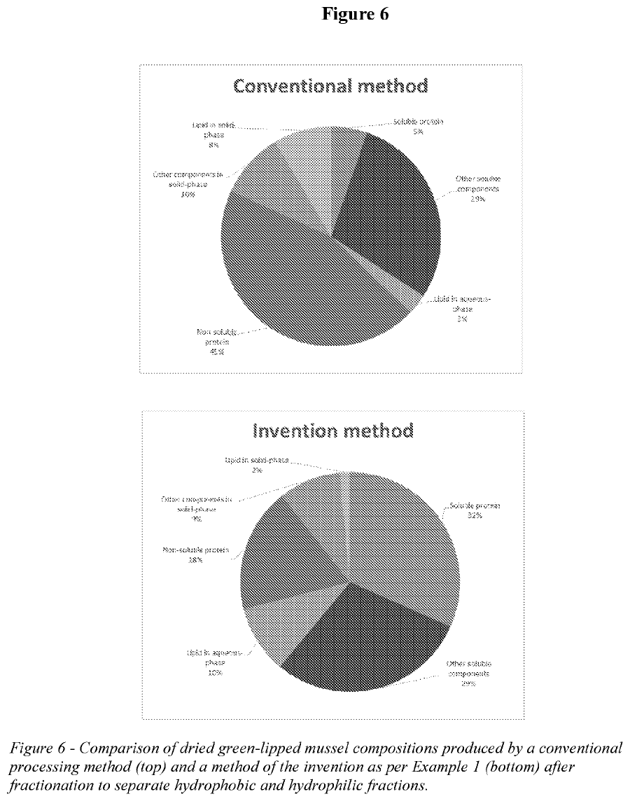

FIG. 6 is a graph showing a comparison of the fractions of a dried green-lipped mussel composition of the invention with the fractions of a typical dried green-lipped mussel composition produced by a conventional process (shell manually opened, meat extracted and homogenised by blending, freeze dried and milled to powder).

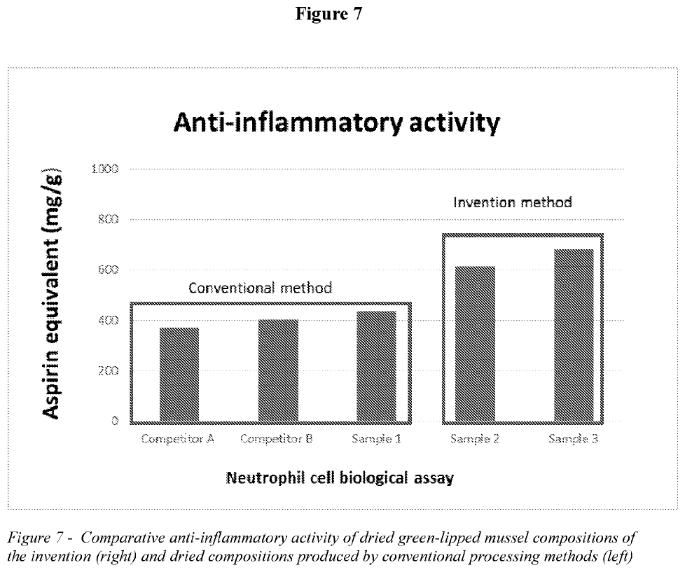

FIG. 7 is a graph showing the results of an anti-inflammatory activity study of two dried green-lipped mussel compositions produced by the process of the invention in comparison with the anti-inflammatory activity of three dried green-lipped mussel compositions produced by conventional processes.

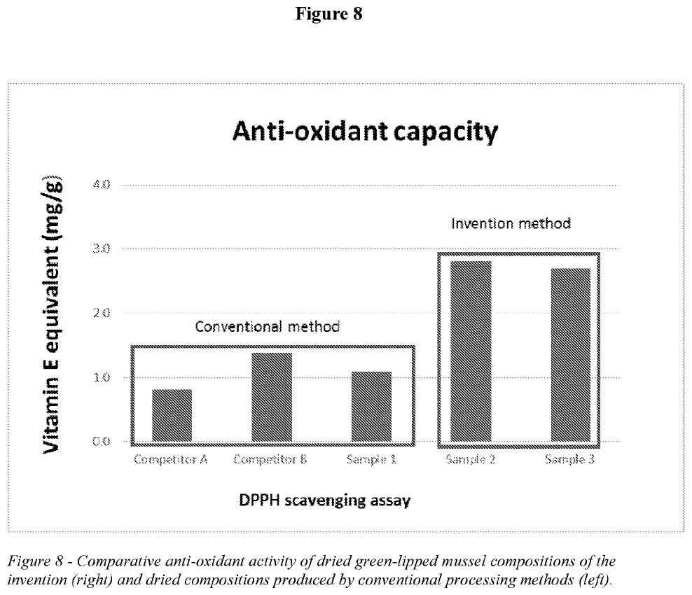

FIG. 8 is a graph showing the results of an antioxidant activity study of two dried green-lipped mussel compositions produced by the process of the invention in comparison with the antioxidant activity of three dried green-lipped mussel compositions produced by conventional processes.

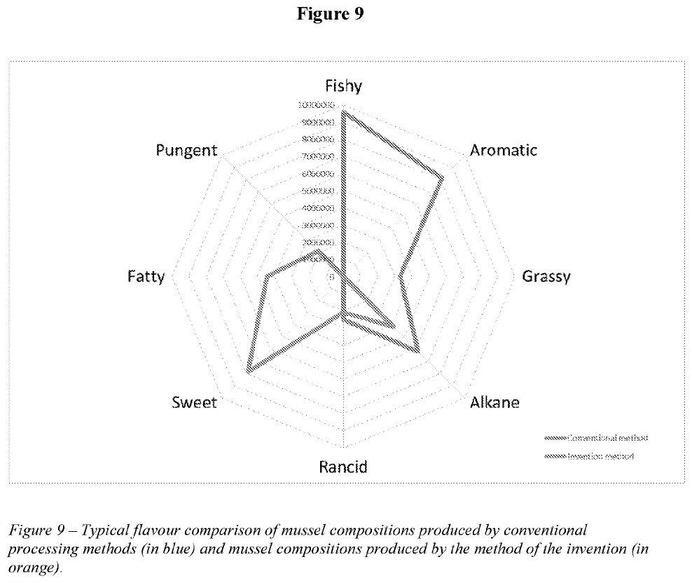

FIG. 9 is a graph showing the flavour profile of a dried green-lipped mussel composition produced by a conventional processing method (shown in blue) and a dried green-lipped mussel composition produced by the method of the invention (shown in orange).

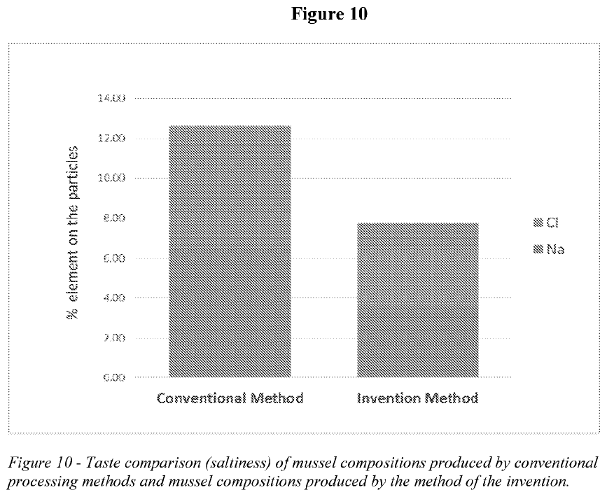

FIG. 10 is a graph showing the taste profile (saltiness) of a dried green-lipped mussel composition produced by a conventional processing method and a dried green-lipped mussel composition produced by the method of the invention.

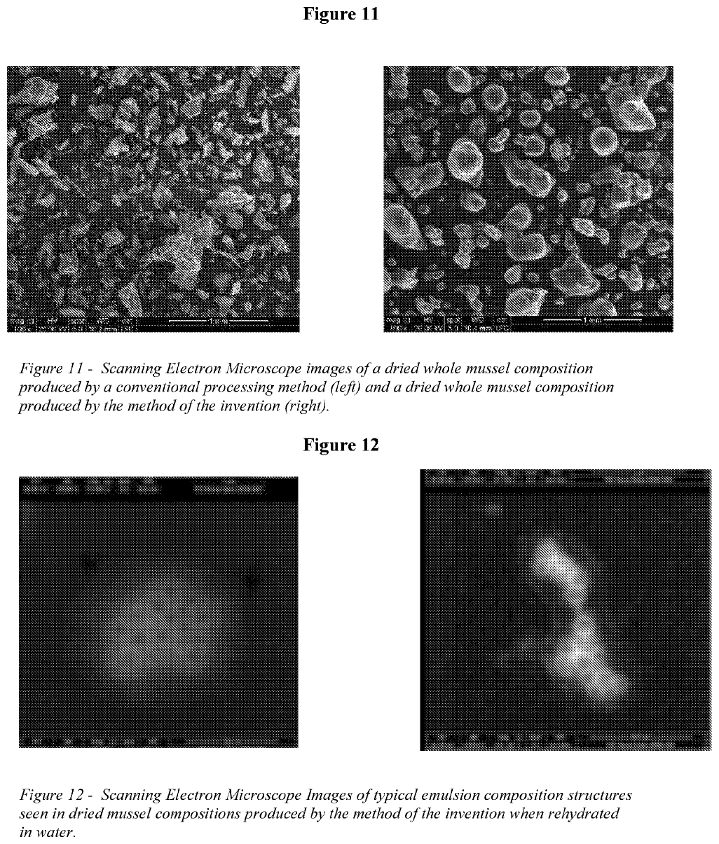

FIG. 11 are some scanning electron microscope (SEM) images collected on FEI Quanta 450 SEM showing the microstructure of a dried green-lipped mussel composition produced by a conventional processing method and a dried green-lipped mussel composition produced by the method of the invention. Samples were fixed on SEM stages and coated with carbon to optimise visualisation.

FIG. 12 are some SEM images showing the typical microstructure of dried green-lipped mussel compositions produced by the method of the invention and rehydrated in water.

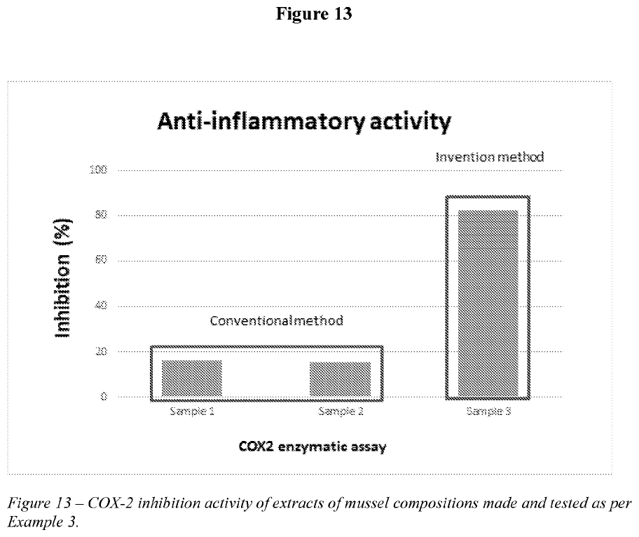

FIG. 13 is a graph showing the COX-2 inhibition activity of mussel compositions made and tested as per Example 3.

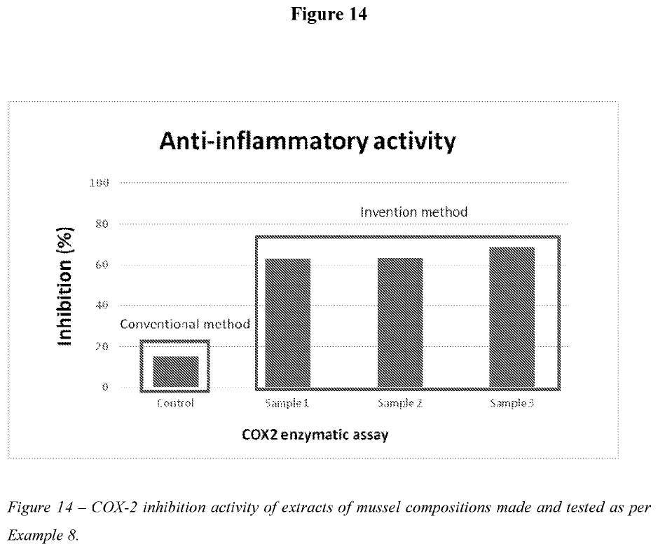

FIG. 14 is a graph showing the COX-2 inhibition activity of mussel compositions made and tested as per Example 8.

The following description will describe the invention in relation to preferred embodiments of the invention, however the invention is in no way limited to these preferred embodiments as they are purely to exemplify the invention only and it is envisaged that possible variations and modifications could be made that would be readily apparent to those skilled in the art without departing from the scope of the invention.

The invention relates to an improved method of processing whole fresh shellfish to obtain high yields of liquid or dried compositions comprising a high concentration or high yield of bioactive components. The invention is directed particularly, but not necessarily solely, towards the processing of whole fresh or live bivalve mollusc species (preferably whole live bivalves) including, but not limited to the following: all mussel species such as the New Zealand green-lipped mussel (Perna canaliculus), the Asian green mussel (Perna viridis), the Mediterranean blue mussel (Mytilus galloprovincialis), the common blue mussel (Mytilus edulus), California mussel (Mytilus californianus), the brown mussel (Perna perna), the Korean mussel (Mytilus coruscus), the Chilean mussel (Mytilus chilensis), the bay mussel (Mytilus trossulus), the ribbed mussel (Geukensia demissa), the date mussel (Lithophaga lithophaga), and the fresh water Zebra Mussel (Dreissena polymorpha); Brachidontes rodriguezii; Perumytilus purpuratus; Aulacomya ater; Choromytilus chorus; Modiolus mussel species, other Perna mussel species; all species of clams; all species of cockles; all species of oysters including rock oysters (Saccostrea glomerata), bluff oysters (Ostrea chilensis) and pacific oysters (Crassostrea gigas); all species of pipi (Phaphies species) including toheroa and tuatua; all species of scallops including Golden Bay scallops (Pecten novaezelandiae) queen scallops (Zygochlamys delicatula); all species of cockles including Austrovenus stutchburyi The invention is further directed to the processing of crustaceans such as scampi (Metanephrops challengeri), crabs, lobster, crayfish, prawns, krill and other crustaceans, as well as other molluscs such as paua (Haliotts species) and echinoderms, particularly sea urchins such as kina (Evechinus chloroticus).

The method of the invention does not require the use of mechanical processes which damage or break up the shellfish material or comminute the flesh of the shellfish prior to processing. Nor does the method require the use of high temperatures. The method involves at least one enzyme treatment step which comprises the application of at least one enzyme formulation to whole fresh (preferably live) shellfish starting material for a sufficient period of time to produce a liquid emulsion-like composition. The enzyme formulation comprises one or more enzymes suitable for acting on one or more target substrates of the shellfish to substantially separate the target substrates from the shells or exoskeletons of the shellfish and to substantially liquefy the target substrates, that is, by reducing or breaking down the target substrates into a liquid emulsion-like composition.

The target substrates can include any biological material present on or in the shells or exoskeletons of the shellfish, for example, the meat or flesh inside the shells or exoskeletons, chitosan present on the shells or exoskeletons, layers of biological material that might be present inside the shells or exoskeletons (for example, the nacre, prismatic and periostracum layers present in mussels (resembling skin), ligaments, abductor muscles, teeth, byssus threads (or beards), gut and feet.

The non-target solid biological material or non-target substrates (for example the shells or exoskeletons and/or fragments thereof and any other non-target material) may then be removed or separated from the liquid composition at the completion of the enzyme treatment step. The removed calciferous shells or exoskeletons are typically very clean since substantially all of the target biological material has been removed from the shells by the enzyme treatment step.

The key to the invention is that the compositions can be produced directly from whole fresh (including live) shellfish starting material without using any mechanical processing methods to reduce the size of the shellfish material or comminute the flesh of the shellfish before application of the enzyme formulation. The application of the enzyme formulation directly to the whole fresh shellfish starting material not only removes all of the biological material from the shells or exoskeletons of the shellfish but also substantially liquefies the biological material. The biological material can be removed from the shells or exoskeletons of the shellfish, homogenised and emulsified, in one step, which does not include mechanical processes to extract the meat, break down or reduce the size of the starting shellfish material. The process is also very fast in comparison to prior art enzyme hydrolysis methods. Whole fresh or live shellfish starting material can be substantially separated from its shells or exoskeletons and liquefied in a single enzyme treatment step in less than 40 minutes. The process of the invention creates shellfish compositions having significant advantages and useful properties as described herein.

It has been found that the method of the invention produces a liquid emulsion-like composition which appears to have the properties of a self-emulsifying composition. The property of self-emulsification permits such compositions to be administered in concentrated form, as for example in a soft-gel or hard-shell capsule form with the expectation that a fine emulsion will be formed in the digestive tract, so that when given orally, there is improved absorption of bioactive compounds. Self-emulsifying compositions when combined with an aqueous medium have improved physical stability when compared with conventional emulsions. Independent tests have been done on the compositions of the invention which show that the compositions have much higher stability than prior art compositions produced by conventional processing methods.

The method of the invention can therefore be used to prepare shellfish compositions which spontaneously self-emulsify upon addition to water or other aqueous media. These compositions permit the delivery of bioactive components in a form which, due to the stability and homogeneity of the resulting aqueous emulsion, will provide good and unexpectedly consistent bioavailability.

The liquid emulsion-like composition is a stable composition having at least two phases, namely a continuous phase and a dispersed phase, wherein at least one phase is a hydrophobic phase and at least one phase is a hydrophilic or aqueous phase, and the composition may also comprise some solid particles in solution or suspension. Preferably the composition comprises a mixture of particle sizes, with a mean particle size distribution of between 0.1-100 μm, and including some micro-particles and/or micro-droplets and/or nano-particles and/or nano-droplets. It has been found that the majority of the particles in the compositions of the invention are micro-particles with sizes in the range of between about 100-50,000 nm, and preferably in the range of about 100-10,000 nm. It has been found that at least some of the particles in the hydrophobic phase have a layer encapsulating or surrounding the particles or droplets or globules wherein one or more lipid or lipophilic bioactive components are located inside the particles or droplets or globules and are protected. The particles or globules may be lipoproteins or similar. The hydrophobic phase is dispersed and/or suspended in the continuous or hydrophilic or aqueous phase. The continuous or hydrophilic or aqueous phase comprises one or more bioactive components dispersed and/or suspended therein, which may include proteins, peptides, amino acids, carbohydrates, vitamins, elements, glycogens, polysaccharides, minerals, taurine, polyphenols, carotenoids, glucosaminoglycans and collagen.

Advantageously, the main steps of the process of the invention are able to be carried out in a single treatment vessel or chamber which substantially separates the biological material from the shells or exoskeletons of the shellfish and liquefies (homogenises and substantially emulsifies) the shellfish starting material without the need for mechanical size reduction processes to be used before the application of the enzyme(s). The process of the invention enables an increased yield of bioactive components to be obtained, in a stable emulsion-like composition. Liquefication of the target biological material of the shellfish is achieved within a very short time with very little waste and very little yield loss.

As shown in FIG. 1, the process of producing a liquid composition from whole fresh (preferably live) shellfish starting material comprises the essential steps of applying at least one enzyme formulation comprising one or more enzymes to at least a portion of the shellfish and leaving the shellfish in contact with the enzyme formulation for a sufficient period of time to separate the target substrates from the shells or exoskeletons of the shellfish, and substantially liquefy the target substrates. The enzyme formulation firstly separates the meat or flesh and other biological material from the shells or exoskeletons of the shellfish and secondly gently and progressively breaks down the biological material into smaller particles, thereby releasing bioactive components including nutrients, functional molecules and bioactive compounds. Any residual non-target materials or substrates, namely solids such as shells or exoskeletons and/or fragments thereof can then be removed from the resulting liquid composition. Preferably the method of the invention includes a warming step to activate the enzymes and speed up the process. The liquid shellfish composition can be dried to produce a dried shellfish composition. The process of the invention can be carried out as a batch process, or advantageously as a continuous or semi-continuous process.

The whole fresh (preferably live) shellfish are preferably cleaned and processed as soon as possible after harvesting, so that the shellfish is processed fresh and preferably alive, or at least within 12 hours, and preferably within three hours post-mortem. The shellfish should be sufficiently cleaned to meet food-grade standards, for example, by removal of all dirt, by-products, other marine organisms and foreign matter from the outside of the shellfish. If it is not possible to process the shellfish quickly after harvesting, the shellfish can be cleaned and stored in cold storage (at about 4-9° C., ideally 7° C.) for up to 48 hours before processing, so that they remain alive. Cold storage may be preferred in some cases because the sea water drains out naturally which is helpful to reduce water content for later drying of the composition.

After harvesting and cleaning, at least one enzyme treatment step (10) is carried out which is designed to remove or separate targeted biological material, for example the meat and/or other biological material present in or on the shells or exoskeletons of the shellfish, from the shells or exoskeletons of the shellfish, and at the same time gently liquefy or reduce the size of the targeted biological material to produce a liquid emulsion-like composition (11). The enzyme treatment step involves the exposure of one or more target biological materials of the shellfish to an enzyme formulation, comprising one or more enzymes that are suitable for acting on the target substrates.

If the shellfish is a species of bivalve, then advantageously whole fresh (preferably live) bivalves can be used in the process of the invention. For processing whole bivalves it is necessary to open or gap the bivalves, or pierce or penetrate at least a portion of the shells in some manner in order to expose at least a portion of the interior of the shells containing the meat and other biological material to the enzyme formulation. This is preferably done by application of gentle heat if the bivalves are alive, or it can be done by an HPP process, or other processes such as laser opening methods localised to the abductor muscle to trigger gapping or opening. If other piercing or cracking methods are used, these are preferably gentle methods which cause minimal disturbance to the biological material inside the shells.

In a preferred embodiment of the invention at least one gentle heating step or warming step is conducted. The warming step may be used for two purposes. Firstly, the warming step can be used to condition the shellfish for the enzyme treatment step. That is, to bring the shellfish up to an optimum temperature for facilitating the enzyme treatment step by activating the enzyme formulation to achieve a faster reaction. Secondly, if whole live bivalve species are being processed, the warming step can be used to at least partially open or create a gap in the shells of the bivalves so that the material inside is exposed to the enzyme formulation. Preferably the bivalves are alive at or up to the point of the gapping or opening step. An enzyme formulation comprising one or more proteolytic enzymes selected to act on the abductor muscles as the target substrate is also preferably used to facilitate the full opening of the bivalves once they are gapped or partially opened by the warming step.

The warming step can be carried out by any means known in the art, for example, by application of a heat source directly or indirectly to the shellfish. In a preferred embodiment of the invention, the warming step is carried out by way of application of steam (e.g. flash steam injection or infusion) at a temperature of about 90-100° C. to quickly achieve a temperature of about 35-55° C. in or around the shellfish. The length of time that the steam is applied for will vary depending on several factors such as the starting temperature of the shellfish, the amount of shellfish being processed, and the type and size of processing equipment used. It is important that the warming step is not carried out for too long, and that the processing temperature is well controlled in order to avoid heat damage to the bioactive components in the shellfish. Warming by flash steam injection or infusion is advantageous because it is very fast. Alternatively, the warming step could be carried out by the use of a thermal jacket or other thermal means to heat the shellfish to the optimum temperature, however this would be a slower process. The process of the invention could include application of more than one heating means or steps, for example, a combination of a flash steam injection and a thermal jacket. For example, the steam injection may be used to warm the shellfish to the optimum temperature, after which the thermal jacket could be used to maintain a specific temperature as required during processing.

The or each enzyme formulation can comprise one or more types of enzymes sourced from animal, plant or microbial origins, or a combination of one or more enzymes with one or more acids or alkalis. All enzymes behave differently and not all enzymes act on the same substrate. Even enzymes in the same general group act on different substrates in a different manner. The activity of enzymes is linked to many factors including temperature, time and pH. Accordingly, the selection of enzymes requires consideration of the species of shellfish used, the target substrates to be acted on, the form of composition desired, the processing equipment used and the factors that will influence and/or facilitate enzyme activity.

Examples of enzymes that may be used in the enzyme formulation include but are not limited to the following: lipase, phospholipase, phosphatase, glycogen phosphorylase, glucosyltransferase, glucosidase, proteinase, collagenase, glycogen debranching enzymes, phosphoglucomutase, cellulases, chitinases, polysccharidases, disaccharidases, alginase, amylase, maltase, peptidase, pepsin, thrombin, trypsin, α-Amylase (from malted cereals), ß-Amylase (from sweet potato or malted cereals), actinidin (from kiwifruit), ficin (from figs), bromelain (from pineapple), papain (from Papaya), and enzymes derived from the following microorganisms: Bacillus amyloliquefaciens, Bacillus subtilis, Bacillus licheniformis, Bacillus coagulans, Bacillus acidopullulyticus, Bacillus halodurans, Aspergillus melleus, Aspergillus oryzae, Aspergillus niger, Lactococcus lactis, Geobacillus stearothermophilus, Rhizomucor miehei, Micrococcus luteus, Penicillium funiculosum, Trichoderma reesei, Trichoderma viride, Escherichia coli, Kluyveromyces lactis, Paenibacillus macerans, Chaetomium gracile, Penicillium lilacinum, Saccharomyces cerevisiae, Bacillus circulans, Kluyveromyces marxianus, Trichoderma harzianum, Disporotrichum dimorphosporum, Humicola insolens, Talaromyces emersonii, Rhizopus delemar, Rhizopus oryzae, Rhizopus niveus, Hansenula polymorpha, Penicillium camembertii, Candida rugosa, Mucor javanicus, Penicillium roquefortii, Rhizopus arrhizus, Cryphonectria parasitica, Streptomyces violaceoruber, Klebsiella pneumoniae, Streptomyces mobaraensis, Lactobacillus fermentum, Actinoplanes missouriensis, Microbacterium arborescens, Streptomyces olivaceus, Streptomyces olivochromogenes, Streptomyces murinus, Streptomyces rubiginosus and Clostridium histolyticum.

Some examples of currently commercially available enzyme products that could be used as or in the enzyme formulation include ALCALASE, PROTAMEX, FROMASE, NEUTRASE, PROMOD 31, P. OCHROCHLORON MTCC 517, LIQUOZYME, SPIRIZYME, PROVIA and CELLIC, VISCOZYME, CELLULASE, CELLIC, CTEC3, ALTERNAFUEL, CMAXTM3, JTHERM, ACCELLERASE TRIO™, MAXATASE, PESCALASE, FLAVOURZYME, ENZIDASE PTX6L, ENZIDASE LIPASE A2 CONCENTRATE, ENZIDASE 899, ENZIDASE PEP1, LECITASE ULTRA, LIPOZYME TL 100 L, AFP, ESP153, FUNGAL LIPASE 8000, HT PROTEOLYTIC CONCENTRATE, FUNGAL PROTEASE CONC 400 and PROTIBOND TGR.

Examples of acids that may be used in the enzyme formulation include: phosphoric acid, sulphuric acid, tannic acid, citric acid, tartaric acid. Examples of alkalis that may be used in the enzyme formulation include: sodium hydroxide, ammonium hydroxide, magnesium hydroxide, potassium hydroxide.

The or each enzyme formulation can be pre-mixed and applied to the shellfish, or each component of the or each enzyme formulation can be applied to the shellfish separately either at the same time or sequentially during the process in one or more enzyme treatment steps.

The type of the or each enzyme formulation used will depend on the type of shellfish that is being processed and the desired nature of the resulting compositions which will lead to selection of one or more substrates to be targeted by the enzyme formulation (target substrates). For example, protease or proteolytic enzymes are preferably used on protein substrates, lipases on lipid substrates, carbohydrases on carbohydrate substrates, and other enzymes on other substrates. Therefore the characteristics of the enzyme formulation can be selected depending on what substrate is desired to be acted on. Combinations of different enzymes can be used to act on any one or more different substrates present in the shellfish starting material and liquefy the various substrates and consequently release an increased amount and variety of functional or bioactive components. Acids and alkalis can be included in the enzyme formulation to achieve an optimum pH for processing and/or to enhance the activity of certain enzymes and/or to act on certain biological components such as chitosan.

The amount of the or each enzyme formulation used depends on the type of shellfish being processed, as well as the operating parameters (e.g. temperature, pH, time and end point) set by the user, and desired product specifications. The amount of each enzyme included in the or each enzyme formulation should be calculated based on the amount of the or each target substrate which can be estimated based on the weight of the whole fresh shellfish raw material. For example, in a 10 kg batch of mussels, there will be approximately 5 kg of flesh or meat and water (with the remaining 5 kg being shells). There is approximately 12% protein in 5 kg of mussels so in order to effectively liquefy the protein component or the protein-based substrates, the amount of the or each proteolytic enzyme included in the or each enzyme formulation would be calculated based on the estimated 12% protein substrate, not based on the mass of the starting shellfish material. Preferably the amount of the or each enzyme included in the or each enzyme formulation is in the range of 0.1-10% calculated based on the estimated amount of the or each target substrate to be treated in the or each enzyme treatment step. Selection of the types, amounts, and ratios of the enzymes used in the enzyme formulation will generally initially be based on the minimum amounts required to firstly substantially remove the target biological material from the shells or exoskeletons, and secondly to substantially liquefy the target biological material at the set operating temperature and pH. It is envisaged that one or more enzyme treatment steps using one or more different enzyme formulations could be carried out to progressively liquefy the range of substrates present in the shellfish starting material. The types, amounts, and ratios of enzymes used in the different enzyme formulation(s) can then be specifically selected for each enzyme treatment step in order to achieve maximum break down or conversion of each target substrate to release further bioactive components or to produce desired end-product specifications. For example, if a different enzyme formulation is used for liquefying the target substrates in the first or a subsequent enzyme treatment step, the different enzyme formulation would preferably comprise one or more enzymes suitable for further hydrolysing or liquefying one or more non-protein target substrates.

Many commercially available enzymes have been tested in the method of the invention and are effective. The selection of enzymes is generally a balance between cost and the overall efficiency of the enzyme formulation at the particular operating parameters used, and taking into account the desired end-product specifications.

In terms of the processing bivalve species, such as green-lipped mussels, it has been found that use of one or more proteolytic enzymes that is able to target the proteins present in the abductor muscles (for example, myofibrillar proteins) is preferred at least initially so that the shells are rapidly fully opened and the biological material inside is exposed to the enzyme formulation. Other types of enzymes can then be included in the same or a different enzyme formulation or applied in the same or a subsequent enzyme treatment step to target other proteins including the flesh and non-protein substrates on or inside the shells.

Enzymes formulations that have been found to be particularly effective in the processing of bivalves, include one or more enzymes selected from the group comprising enzymes derived from bacterial strains that produce subtilisin, including Bacillus amyloliquefaciens; enzymes derived from Bacillus licheniformis, Bacillus subtilis, Aspergillus niger and Aspergillus oryzae; cysteine proteases; carbohydrases, sucrase, amylase, lipase, phospholipase, phosphatase, esterases, and catalase.

In one preferred embodiment of the invention the enzyme formulation comprises a combination of at least two enzymes selected from the group comprising enzymes derived from Bacillus amyloliquefaciens, enzymes derived from Bacillus licheniformis, cysteine proteases, and enzymes derived from Aspergillus oryzae, wherein at least one of the enzymes is a proteolytic enzyme.

Preferably the ratio of the or each enzyme included in the or each enzyme formulation is in the range of 0.1-10% calculated based on the estimated amount of the or each target substrate desired to be acted on in the or each enzyme treatment step. If an enzyme derived from Bacillus amyloliquefaciens is used, the preferred concentration is between 1-10% of the target substrate (protein substrates), and more preferably between about 5-10%. If an enzyme derived from Bacillus licheniformis is used, the preferred concentration is between about 0.5-6% of the target substrate (protein substrates) and more preferably between about 3-6%. If a cysteine protease such as papain is used, the preferred concentration is between about 0.2-2% of the target substrate (proteins and peptides) and more preferably between about 0.5-1%. If an enzyme derived from Aspergillus oryzae is used, the preferred concentration is between about 0.5-6% of the target substrate (in this case peptides, lipids and/or carbohydrates) and more preferably between about 3-6%.

The optimum pH for processing shellfish is in the range of pH 2-9, preferably about pH 4 to 8, although some enzymes may work at a lower pH. The pH can be adjusted as and when necessary during the process by the addition of a suitable acid or alkali.

The one or more enzyme treatment steps are preferably carried out under temperature controlled conditions and for a specified time period. The reaction temperature is preferably no more than 60° C., and is preferably in the range of about 20-60° C. The total reaction time is preferably less than 120 minutes, more preferably less than 90 minutes and more preferably is in the range of 15-40 minutes. The reaction temperature and duration should be calculated based on the desired end-product specifications. The reaction time is generally set based on the minimum time required to achieve the desired end-product specifications. It has been found that duration of between about 15-40 minutes for each enzyme treatment step is sufficient for achieving a significant degree of hydrolysis. A key advantage of the invention is that a substantially liquid composition can be produced very rapidly from whole fresh shellfish starting material.

The enzyme treatment step(s) may be tailored to suit specific shellfish species. It may be carried out by manually dosing the shellfish with the enzyme formulation(s), or by way of an automatic dosing or dispensing system (as described further below).

The process may further comprise an agitation step during and/or after the warming step and/or the enzyme treatment step, which enables the shellfish to be continually moved and therefore more evenly exposed to the increased temperature and/or the enzyme formulation. Continuous agitation or movement causes the shellfish to have better exposure to the enzyme formulation so that the formulation is distributed widely and evenly over the shellfish, and in the case of bivalve species, inside the gapped shells.

After the enzyme treatment step(s) (10), the resulting composition is in the form of a liquid composition (typically of slurry like consistency) that resembles an emulsion or a colloid (11). The liquid composition is then subjected to at least one separation step (12) to remove any shells, shell fragments, exoskeleton fragments or other large non-target solid biological material from the composition. The separation step can be carried out by any means known in the art, for example, by the use of screens, filters or sieves, or a combination thereof. A series of separation steps may be carried out to obtain a liquid composition with a desired particle size or particular food matrix, or for better recovery of certain bioactive components. Preferably this liquid composition is then siphoned or drained off or otherwise recovered, and the remaining material is retreated with one or more enzyme formulations comprising the same or different enzymes in one or more subsequent enzyme treatment steps (14) so that the larger particles are further liquefied until the desired particle size or particular food matrix or certain level of bioactive components are achieved in respect of substantially all of the shellfish starting material. This means that there is minimal waste and minimal yield loss from the process. It is envisaged that substantially all of the biological material from the shellfish starting material could be liquefied and emulsified in the process of the invention, with only clean residuals of shells and exoskeletons being separated out and discarded or used as a by-product for other applications. One or more further agitation steps could be employed if required, for example, before or after the separation step in order to further homogenise the resulting liquid composition.

In a preferred embodiment of the invention, the main steps of the process are carried out in a single treatment vessel or chamber, as shown in FIG. 2. The treatment chamber (20) is preferably a cylindrical shaped vessel which can be sealed and pressurised. An advantage of using a pressurised vessel is that it could be used to open or gap the shells of whole live bivalve species in an HPP process (with pressure ranging from 200-350 MPa) prior to application of the enzyme formulation to the shellfish. If an HPP process is used to open or gap the shells of bivalves, then any warming step would most likely be carried out by a thermal jacket rather than steam, as steam would not be required to open or gap the shells. However, if warming by steam was able to be well controlled during the process then this may still be the preferred heating method since it requires less expensive equipment and is quicker and easier than HPP. If HPP is used, a warming step may be employed during or after the HPP process to condition the shellfish for the enzyme treatment step.

For batch processing, any amount (e.g. kilograms or tonnes) of whole fresh shellfish may be placed or conveyed from a hopper or other storage container into the treatment chamber (20) for processing. The treatment chamber may have a built-in weigh cell so that the size of each batch can be controlled and monitored. This may not be necessary for continuous or semi-continuous processing methods. The amount of shellfish processed at any one time in the treatment chamber will depend on the internal size and volume of the treatment chamber. There will need to be some void space within the treatment chamber after the shellfish has been added to hold the heat/steam and to give space for movement.

In a preferred process of the invention the treatment chamber is orientated horizontally, rather than vertically, or it is orientated in a sloped position. This avoids any need for a mechanical crushing step, and also avoids the need to add any water to the treatment chamber. If a steam injection is used as a heating means, then it is possible to carry out the process of the invention without adding any other water to the treatment chamber. Advantageously the process of the invention can be carried out without the addition of any water, or with very little water added during the process. This is advantageous as it reduces the costs and time associated with drying the resulting liquid composition, while also having obvious environmental benefits. Whether or not any water needs to be added will depend on the type of shellfish starting material, and the type of processing equipment being used.

The treatment chamber (20) may include at least the following features and components: a sealable opening (21); a heating means (22); a dosing system for the enzyme formulation (23); and an agitating means (24).

The sealable opening (21) can be used both for introduction of the shellfish into the treatment chamber, and for discharging the resulting liquid composition after the enzyme treatment step(s). Alternatively, the treatment chamber could include a separate discharge port if desired especially if the process was continuous or semi-continuous.

The heating means (22) may be a steam heating device such as a flash steam injector or infuser located inside the treatment chamber. Preferably the steam injector or infuser is located in a position within the chamber to enable the steam to be delivered into the central part of the chamber. In another option, the heating means can include a heating element or thermal jacket or heat exchanger (25) located on or near at least one of the walls of the treatment chamber, in place of or in combination with the steam injector or infuser. The heating means is preferably operated to raise the internal temperature of the treatment chamber to between about 35-60° C. in order to condition the shellfish for the enzyme treatment step, by bringing the treatment chamber to an optimum temperature to activate the enzyme formulation, and in the case of whole live bivalves, to cause the bivalves to partially open or gap so that the enzyme formulation can be distributed inside the shells. If a flash steam injection or infusion is used as the heating means, then preferably steam is injected at a temperature of about 90-100° C. for a predetermined time period (generally very short, for example between about 90-120 seconds) in order to quickly but gently raise the internal temperature of the treatment chamber to the optimum temperature. The steam injection or infusion time is dependent on infeed raw material temperature, the size of the treatment chamber, the type of equipment (e.g. whether another heating source such as a heating jacket is also used and if so, whether this is on or off), the efficiency of agitation, the nozzle size of the steam injector or infuser and the volume of steam injected.

The dosing system (23) may include an automatic dispensing device to which the enzyme formulation(s) can be added, which is connected to a dosing means (26) located inside the treatment chamber, so that dosing can be controlled. Preferably the enzyme formulation is poured or sprayed onto the shellfish by way of the dosing means (26) which may have or comprise for example a spray nozzle to facilitate distribution of the enzyme formulation onto the shellfish. Preferably the dosing means (26) is positioned in such a manner to enable substantially even distribution of the enzyme formulation onto the shellfish. The enzyme formulation can be added either before, at the same time, or after the warming step is commenced.

Preferably the treatment chamber (20) or the contents of the treatment chamber are able to be continuously or semi-continuously rotated or agitated. This provides for more effective distribution of heat and enzyme formulation to the shellfish. The treatment chamber preferably comprises an agitating means which can be located inside or outside of the treatment chamber and can include any means that is able to move and/or rotate the contents of the vessel, preferably in a continuous or semi-continuous manner, and in a wide variety of angles or positions to achieve an even and maximum distribution of heat and enzyme formulation onto and around the shellfish. For example, the entire treatment chamber itself may be able to rotate or tumble, preferably in any direction (i.e. both clockwise and anti-clockwise) and preferably the speed of rotating or tumbling can be adjusted depending on the shellfish species and the end product specifications or requirements. Alternatively, the chamber may comprise internal means (24) as shown in FIG. 2 such as rotatably mounted controllable paddles or fins or a tubular tumble member adapted to continuously or semi-continuously rotate or mix the contents of the treatment chamber.

The treatment chamber may also include a recycling system (27) whereby the contents of the vessel can be re-circulated. For example, the enzyme formulation could be re-circulated and re-used, or the liquid shellfish material can be re-circulated during the enzyme treatment step(s) or recycled for subsequent enzyme treatment steps (using enzyme formulations comprising the same or different enzymes) to be carried out after some of the liquid composition is siphoned off or removed. The recycling system may be a pipe or circulation tube extending from an outlet (29) at or near the base of the treatment chamber and providing a fluid pathway back to the dosing means (26) or other suitable inlet port which may be located at or near the top of the treatment chamber.

The internal temperature of the treatment chamber may be monitored by an external temperature gauge or the like (connected to an internal temperature probe) so that the temperature is maintained at the ideal processing temperature (less than 60° C., preferably between 55-60° C.) for the duration of the enzyme treatment step(s). The temperature is maintained by either the heating source (25) being set at the desired temperature for the duration of the reaction time, or by applying further direct flash steam through the steam injector or infuser as and when necessary to maintain the optimum internal temperature. If steam is used to maintain the reaction temperature, then the steam heating means should be capable of being precise and well controlled to avoid overheating the shellfish material.

The duration of the first enzyme treatment step is determined by the amount of time it will take to substantially remove or separate the target biological material from the shells or exoskeletons of the shellfish, and substantially liquefy the target biological material with the selected enzyme formulation. Generally the enzyme treatment step/liquefying process will take less than 120 minutes in total and more preferably less than 90 minutes in total, however in order to achieve complete release or break down of some bioactive components, a longer time period may be required. Preferably though, the duration of the or each enzyme treatment step is between about 15-40 minutes. The reaction time will also be dependent on the type of shellfish species being processed, the size of the batch or amount of shellfish present in the chamber during the enzyme treatment step(s), the type(s) and ratio of enzymes and other additives used (i.e. the nature of the enzyme formulation), the amount of agitation, and the selected processing temperature.

The treatment chamber may comprise an exhaust system (28) which is activated at the conclusion of the enzyme treatment step, that is, the treatment chamber is stopped or deactivated and the exhaust is opened to expel the heat or steam and pressure within the treatment chamber. There may be a window located in the treatment chamber so that operators can check and observe the contents of the chamber at any time during the process.

After the first enzyme treatment step is completed, the target biological material of the shellfish starting material will have been reduced to a predominantly liquid composition, in the form of an emulsion-like composition or colloid (typically of a slurry like consistency), comprising some solid material such as shells and shell fragments and exoskeleton fragments, and other solid biological material comprising non-target substrates, for example, byssus threads (if undesired). The liquid composition is discharged from the treatment chamber (via the sealable opening or other discharge port) and is subjected to at least one separation step (12). The first separation step is carried out to remove residual shells and/or shell and exoskeleton fragments, and any other large solid non-target material from the liquid composition. The clean shells or exoskeleton pieces may be collected in a sieve or other filtration device and discarded, or removed by conveyor into a container. The residual shells, exoskeleton pieces and other undesired waste material may be further processed into other commercial products (for example liquid or dried products for use as pet foods or in animal feeds, or beard/shell products for industrial uses). Alternatively, some of the separated material may be retreated with one or more different enzyme formulations in one or more subsequent enzyme treatment steps if certain bioactive components are desired to be released from this material.

After the separation step, a series of filtration steps (13) may be conducted to progressively reduce the particle size of the liquid composition, by means known in the art, for example, by the use of one or more screens, filters or sieves, with openings of progressively decreasing diameter. Preferably at least one filtration step is carried out after the separation step in order to remove (and potentially recycle) any large particles from the liquid composition after the solids have been removed. Preferably the liquid composition is able to be filtered down to a particle size of less than 200 μm in one filtration step after the separation step. A particle size of less than 200 μm is advantageous if spray drying is used to dry the composition. If freeze drying or other drying methods are employed, particle size may not be so important and the liquid composition could be dried directly after the separation step.

The material remaining (that is, the non-target biological material) after the separation step and/or after the first filtration step (including any remaining active enzyme formulation) may be added back into the treatment chamber (by a recycling system or otherwise) and subjected to one or more further enzyme treatment steps (typically using a different enzyme formulation) to progressively liquefy and emulsify other substrates in the remaining material, in order to release further bioactive components.

The liquid composition may be stabilised (15) before or after the separation or filtration step(s) (if carried out), in order to deactivate the enzyme(s) and to pasteurise or sterilise the liquid composition to meet food safety requirements. Deactivation of the enzyme(s) can be achieved by a number of means known in the art, for example by application of flash heat treatment (such as UHT, HTST, PEF), or by altering the pH of the liquid composition to a pH at which the enzyme(s) become deactivated (i.e. pH<4 or pH>10), for example, by addition of tartaric acid or other acids. Because pH stabilisers could adversely affect some of the bioactive components in the liquid composition or result in separation/denaturation of some components, the preferred stabilisation method is rapid heat treatment. For example, a heat exchanger may be used to quickly increase the temperature of the composition to above 80° C. for a short time period (for example up to 85° C. for 5-15 minutes). Alternatively, a further steam injection or infusion (controlled by a temperature probe) could be applied at the end of the enzyme treatment step to increase the internal temperature of the treatment chamber to this level before the exhaust is activated. Other methods of stabilisation not involving heat treatment may be used such as microfiltration or ultrafiltration methods.

The resulting liquid composition can be used as is, or it can be dispensed into containers and stored at low temperature for later use, or it can be immediately frozen for later use. If the liquid composition is frozen immediately an enzyme deactivation step may not be required, however an enzyme deactivation step may be applied upon thawing.

In FIG. 1, a drying step (16) is carried out after the separation step (12) in order to produce a dried composition. The liquid composition can be dried immediately using any known drying methods in the art. Preferably drying is carried out by low temperature (<80° C.) drying means such as freeze drying, or by flash drying means such as spray drying, vacuum drying or belt drying. After drying, the dried composition may be ground or milled into a powder (17) by methods known in the art. The liquid or dried compositions can be further processed by one or more separation, fractionation or extraction steps (18) to produce various product formats.

FIG. 3 provides a more detailed schematic representation of a preferred method of the invention, including the use of a treatment chamber as shown in FIG. 2 and including subsequent possible processing steps.

The process of the invention provides an improved method for large scale commercial processing of shellfish species in order to obtain high yields of compositions with high yields of bioactive components. The process of the invention is able to convert whole fresh (including live) shellfish, into a high quality liquid or dried composition within a very short time frame. The number of processing steps is significantly reduced in comparison to conventional methods, with consequent reduction in processing time and costs. Furthermore, the risk of contamination and oxygen exposure are greatly reduced, especially if a single treatment chamber as described is used to carry out the main steps of the process. The process of the invention produces a very high yield of product in comparison to conventional methods. For example, the dry yield recovery from the processing of whole live Perna canaliculus is about 20-40% higher than that achieved from other conventional processes. The process of the invention generally yields about 45-50% of the whole live shellfish starting material. For example, if 400 kg of green-lipped mussels are added to the treatment chamber at the start of a cycle, on discharge there will be about 200 kg of discarded shells and shell fragments, and about 200 L of liquid composition.

The compositions of the invention have an increased yield of bioactive components and an unexpected and highly desirable microstructure which is expected to increase the bioavailability of the bioactive components. Advantageously, no antioxidants are required to be added during or after processing in order to maintain the bioactivity of the compositions. Furthermore, no surfactants, co-surfactants or emulsifiers are required to be added to the compositions in order to maintain the stability of the compositions.

### Compositions of the Invention

The process of the invention produces compositions containing high yields of bioactive components in unique food matrices in the form of emulsion-like compositions. The process firstly removes or separates the target biological material from the shells or exoskeletons of the shellfish and then continuously liquefies the biological material into smaller biologically viable components or particles, without using any mechanical methods, thereby preventing any significant damage or destruction to beneficial bioactive components present in the shellfish starting material. The emulsion-like compositions are stable in either liquid form or dried form, and they comprise uniformly distributed particles, droplets and/or globules or biological molecules of reasonably uniform size and shape (as shown in FIGS. 4 and 5 relating to Example 1 below and in FIGS. 11 and 12). The compositions have been shown to exhibit higher levels of bioactivity than existing products. No anti-oxidants, surfactants, co-surfactants, emulsifying agents or other additives are required to be added to the compositions in order to maintain the bioactivity or to otherwise stabilise the compositions. The compositions are therefore completely natural.

One of the key advantages of the compositions of the invention is that due to the unique emulsion-like structures or food matrices the bioactive components are more easily or readily absorbed into the body (via cells or bloodstream or skin or other tissues), therefore the compositions of the invention have improved bioavailability and will more effectively and consistently deliver beneficial bioactive components. Without being bound by theory, the inventor believes that the method of the invention causes a natural process of self-emulsification to occur which means that the bioactive components present in the compositions of the invention will be easily absorbed into the body via paracellular absorption between cells. Literature suggests that self-emulsifying formulations, when given orally, may offer improvements in both the rate and extent of absorption of the bioactive compounds present in the composition and also the consistency of the resulting plasma concentration profiles. The bioactive components of the compositions of the invention will therefore be delivered much more effectively than compositions of the prior art. Furthermore, the property of self-emulsification permits the compositions of the invention to be administered in concentrated form, as for example in an encapsulated format, with the expectation that a fine emulsion will be formed in any targeted location in the digestive tract.

Without wishing to be bound by theory, it is believed that the process of the invention releases physiochemical components which naturally function as surfactants and/or co-surfactants and thereby act as natural solubility enhancers in a self-emulsifying system or similar. The lipid or lipophilic bioactive components present in the hydrophobic phase of the composition are dispersed in a stable and homogenous manner through the continuous or hydrophilic or aqueous phase. It has been found that at least some of the particles in the hydrophobic phase have a layer surrounding or encapsulating the particles or droplets or globules wherein one or more lipid or lipophilic bioactive components are located inside the globules and are protected by the surrounding layer. It is possible that these particles or globules are lipoproteins or similar. The continuous or hydrophilic phase of the composition has one or more bioactive components dispersed or suspended therein, and may also comprise some solid particles in solution or suspension.

It is likely that the components that function as surfactants and/or co-surfactants and/or emulsifiers comprise low molecular weight proteins and/or peptides as these appear to remain on the surface of the particles or globules that are dispersed through the hydrophilic phase. These substances could assist in forming the structured particles or globules which then repel each other and the repulsive forces cause them to remain stably suspended in the hydrophilic phase. Alternatively, or additionally it may be that the substances modify the viscosity of the composition which could help to create and maintain the suspension of the hydrophobic particles or globules in the hydrophilic phase.

A specific advantage of the liquid composition of the invention is that it comprises a high percentage of material of small particle sizes in comparison to comparative products produced by conventional processing methods. For example, in one study, the inventor found that a green-lipped mussel product produced by the method of the invention had about 60% of 40-50 μm sized particles, in comparison to comparative products produced by conventional processes which had a majority of particles sizes in the range of 300-1200 μm. There is also a much higher concentration of particles or globules in the aqueous phase of the compositions produced by the method of the invention compared to compositions produced by conventional processing methods. Other studies have shown that the majority of particles in the compositions of the invention are micro-particles with sizes in the range of about 100-50,000 nm, preferably between about 100-10,000 nm.

FIG. 11 shows images obtained from an FEI Nova NanoSEM 450, high resolution, field emission gun scanning electron microscope (FEG-SEM), of the microstructure of a dried green-lipped mussel composition produced by the invention in comparison to one produced by a conventional processing method. The images show a clear distinction in particle shape and size. The compositions produced by the method of the invention have much smaller and more uniform particle structure and size, comprising structured spherical shaped particles. FIG. 12 shows typical images of the emulsion-like structures of dried green-lipped mussel compositions produced by the method of the invention reconstituted with water. The rehydrated compositions produced by the invention are stable in aqueous suspension and form unique microstructures comprising structured spherical biomaterials comprising both lipids and proteins, which could be lipoproteins or particles comprising lipid bilayers or similar. The images show that the compositions comprise a mixture of material of varying size including some nano-particles or nano-droplets and some micro-particles or micro-droplets.

It is believed that lipids and/or lipophilic bioactive compounds are encapsulated and protected inside the structured particles in the hydrophobic phase, and other bioactive compounds are dispersed or suspended in the hydrophilic phase.

Due to the small uniform particle size achieved by the process of the invention (e.g. typically between about 0.1-50 μm for a green-lipped mussel composition), various drying methods are possible, including spray drying. It is not generally possible to use spray drying to produce dried mussel compositions after conventional processing, because the resulting compositions have material particle sizes over ten to hundred times higher which are difficult to effectively spray dry using standard spray drying equipment.

The stable nature of the liquid compositions of the invention also allow for other direct non-thermal sterilization processes, such as Pulsed Electric Field (PEF) or Ultra-High Temperature (UHT), or High Temperature/Short Time (HTST) pasteurization to be used if desired. The unstable and non-uniform unstructured compositions prepared by conventional processes make it difficult to use these high efficiency non-thermal sterilization methods.

A further advantage of the invention is that the processing method produces, releases or frees up more amino acids and small proteins and/or peptides, some of which are essential amino acids, some of which are flavour enhancers and some of which are functional amino acids and peptides. The inventor has found that the compositions of the invention have improved sensory attributes including smell, taste or flavour profiles due to the increased amount of flavour enhancing amino acids. See FIGS. 9 and 10. FIG. 9 shows that dried green-lipped mussel compositions of the invention have a less fishy and grassy flavour profile than prior art compositions, and that they have a sweeter flavour profile. FIG. 10 shows that dried green-lipped mussel compositions of the invention have lower levels of sodium and chloride on the particle surfaces and are therefore less salty. This is thought to be due to the enzyme treatment process in that during enzyme hydrolysis the sodium connects with other amino acids that are released during the process that are not released through prior art processing methods.

### Further Processing of Liquid and Dried Compositions

The liquid or dried compositions of the invention can either be used as is, or be formulated into other finished products in various dosage formats including oral dosage formats, topical dosage formats and other dosage forms for various uses as described below.

The liquid and dried compositions of the invention can be used as is or formulated for use in a wide variety of purposes including as foods, food supplements, food ingredients for use in food applications, or for cosmetic, pharmaceutical or nutraceutical applications, or veterinary applications. Alternatively, the liquid and dried compositions of the invention can be used or sold as intermediate products intended for further processing into any number of different extracts and/or product formats which could again be used in a wide variety of applications including food applications, pharmaceutical or nutraceutical applications, cosmetic applications, veterinary applications etc. The stable and uniform compositions produced by the process of the invention make them desirable for further processing to obtain extracts and other product formats that are expected to have high levels of bioactive components and improved bioavailability.

The compositions of the invention may be formulated into food products, dietary supplements, nutraceutical compositions, veterinary compositions, pharmaceutical compositions or cosmetics. A variety of dosage forms are possible including oral dosage forms such as tablets, capsules, dried powder formats, oils, food ingredients; topical dosage forms for external use such as creams, gels, emollients, ointments, lotions, dressings such as plasters, bandages and medicated dressings; and other internal dosage forms including injectable forms.

Both the liquid and dried compositions of the invention can be subjected to one or more fractionation, separation or extraction steps to yield different useful products. For example, the compositions can be further separated into various fractions, including but not limited to a hydrophobic or lipid-rich fraction, and a hydrophilic fraction containing water soluble proteins, peptides, amino acids, nucleic acids, minerals, carbohydrates, vitamins, biotin and others, and water insoluble (high molecular weight materials) and undissolved proteins etc.

Separation and/or fractionation of the liquid composition can be achieved by any methods known in the art, for example, ultrafiltration, Nano filtration, siphoning or pumping off the fatty layer or fat or lipid fraction or emulsion layer, screen filter separation of the liquid from the solids, centrifugation, decanting, tricanting and/or water or solvent extraction methods. Separation and/or fractionation of the dried composition can be achieved by any methods known in the art, however, in relation to the dried composition, solvent extraction methods to remove the lipid-rich fraction from the hydrophilic fraction, are preferred. This is due to the nature and structure of the dried compositions of the invention which have good extractability characteristics.

The lipid rich and/or hydrophilic extracts can also be formulated into many different types of formats and products as described above and below. Due to the increased yield of bioactive components in the liquid and dried compositions of the invention, it is putated that any extracts produced therefrom will have increased concentrations of bioactive components with improved bioavailability.

### Examples of Possible Product Formats

It is envisaged that the following product formats (non-limiting) could be derived from further processing of either the liquid or dried compositions of the invention:
Oils—in liquid form (including encapsulated form in either hard-shell capsule form or soft gel form), dry form including tablets or powders, or oil with carrier form, for use in dietary supplements or pharmaceutical or nutraceutical products, cosmetics or veterinary products;
Liquids (either whole mussel compositions (including the lipid rich fraction), or fractionated mussel liquids (with the lipid rich fraction removed leaving only the hydrophilic fraction (comprising both water soluble and non-water soluble fractions, or water soluble fraction only) for use in dietary supplements or pharmaceutical or nutraceutical products or cosmetics or veterinary products; packed in any desired format (for example, syrups, elixirs, cachets, encapsulated form in either hard-shell capsule form or soft gel form etc).
Powders (either whole mussel powders (including the lipid rich fraction), or fractionated mussel powders (with the lipid rich fraction removed leaving only the hydrophilic fraction (comprising both water soluble and non-water soluble fractions, or water soluble fraction only) for use in dietary supplements or pharmaceutical or nutraceutical products or cosmetics or veterinary products; packed in any desired format (for example, capsules, tablets, sachets etc).
Food ingredients in any desired format for use as food flavourings, seasonings, in ready made sauces, meals etc.
Pet foods

### Example 1

Green-lipped mussels (Perna canaliculus) were processed according to the method of the invention. 60 kg of live whole mussels were added to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The chamber was then opened and 6% of an enzyme formulation (based on the total protein amount of between 3-4 kg) was manually added (in liquid form). The enzyme formulation comprised a protease enzyme derived from Bacillus species (Bacillus licheniformis commercially available as ESP153). The internal temperature of the treatment chamber was maintained at about 55-60° C. by the initial steam injection (no further steam was required). The chamber was rotated for a period of about 40 minutes. At the end of this time period the chamber was deactivated by activation of the exhaust which expelled the heat and pressure from the chamber. The contents of the chamber were then discharged onto a separating screen to remove any residual shells, shell fragments and any other large particles. The liquid composition was then filtered through a 200 μm mesh filter. FIG. 4 shows a microscopy image and associated graph of the particle size distribution of the resulting liquid composition of green-lipped mussels produced in this example. The particle sizes range from 1 μm to 100 μm, with the majority of particle sizes being between 1 μm-50 μm, and a high concentration of particle sizes between 1 μm-10 μm. The liquid composition comprises an emulsion-like structure of suspended particles (in an aqueous medium) of uniform size and distribution. The emulsion could be a double or multiple emulsion comprising low molecular weight proteins/peptides and other substances which appear to remain on the surface of the oil/water droplets and assist in stabilising the composition.

After the separation and filtration steps, the liquid composition was dried by freeze drying, without any stabilisation step. FIG. 5 is a graph showing the particle size distribution of the dried green-lipped mussel composition produced in this example of the invention. The dried composition is rehydrated. The particle sizes range from 1 μm to 100 μm, with the majority of particle sizes being between 1 μm-50 μm.

About 45-50% yield of liquid composition was obtained in this example (i.e. about 25-30 L of liquid composition). From that, about 6-7% yield of dried composition was obtained (i.e. about 3-4 kg). The dried composition had a moisture content of less than 6%. The dried composition was highly soluble and could be readily rehydrated in aqueous solution to achieve a stable composition substantially the same as the original liquid composition (as shown in FIG. 4).

FIG. 6 provides an analysis of the main components of the dried green-lipped mussel composition produced by the process described in this example. As shown in FIG. 6, the components of the composition produced by the process of the invention are different to that of a dried composition produced by a typical conventional process (in the comparative conventional process live mussels were manually opened, the meat or flesh removed and minced and then freeze dried). The dried composition produced in this example comprises about 10% lipids, 32% proteins and 29% other soluble components in its aqueous phase, and about 2% lipids, 18% non-soluble proteins and 9% other non-soluble components. In comparison, the dried composition produced by conventional process comprises about 3% lipids, 5% proteins and 29% other soluble components in its aqueous phase, and about 8% lipids, 45% non-soluble proteins and 10% other non-soluble components. The compositions of the invention comprise a much higher amount of soluble protein in the aqueous phase.

Given that the composition of biological material present in mussels varies between seasons, the dried compositions of the invention are likely to comprise about 7-16% lipids and 45-55% protein. Advantageously, it is expected that a dried composition of the invention could comprise >85% of soluble proteins and other soluble components in its aqueous phase. In comparison, the dried composition produced by conventional processing comprises typically only about 25% of soluble proteins and other soluble components in its aqueous phase.

FIG. 6 shows that there is over 70% hydrophilic fraction in the composition of the invention which is almost double the 37% hydrophilic fraction in the composition produced by conventional processing methods. This demonstrates the very high yields of emulsion-like-composition obtained by the process of the invention, which have more bioactive components in a highly bioavailable form. It is envisaged that the non-soluble portion could be further processed in the process of the invention using one or more different enzyme formulations to break down or convert the non-soluble material to release further bioactive and soluble components.

### Bioactivity Studies

The dried composition of Example 1, together with one other dried composition that was prepared in the same way but dried by spray drying rather than freeze drying, were tested for their anti-inflammatory properties, in comparison with three other dried mussel extracts which were prepared by conventional processing methods.

The relative anti-inflammatory properties of the test samples were determined by establishing their abilities to inhibit the activation of neutrophils as measured by the production of superoxide. The efficacy of the test samples was referenced against Aspirin, as well as an un-supplemented control group.

Details of the test samples and the methods used to make them are set out in the following table:

| Sample No. | Description |
|---|---|
| 1 | Competitor A (Whole dried green lipped mussel extract |
| | prepared by conventional process of mechanical crushing |
| | and homogenising followed by drying) |
| 2 | Competitor B (Whole dried green lipped mussel extract |
| | prepared by conventional process of mechanical crushing, |
| | centrifuging followed by freeze drying) |
| 3 | Sample 1 (Whole dried green lipped mussel extract |
| | prepared by conventional process of manually opening |
| | mussels, blending the meat, and freeze drying) |
| 4 | Sample 2 (Whole dried green lipped mussel extract |
| | prepared by method of invention (with freeze drying)) |
| 5 | Sample 3 (Whole dried green lipped mussel extract |
| | prepared by method of invention (with spray drying)) |

Samples 1, 2 and 3 (actually numbered samples 3, 4 and 5 in the above table) were produced from the same batch of mussels. Each of the above test samples was extracted with ethanol at a ratio of 1:10 (w:v) and the residues were then extracted with distilled water at the same ratio, so that the activity of both the lipid rich or hydrophobic fraction and the hydrophilic or aqueous fraction of each of the test samples could be tested. The experimental procedure for determining the effects of the test samples on inflammation was based on the methods described in Tan, A S and Berridge, MV (2000). Superoxide produced by activated neutrophils efficiently reduces the tetrazolium salt, WST-1 to produce a soluble formazan: a simple colorimetric assay for measuring respiratory burst activation and for screening of anti-inflammatory agents. J Immunol. Meth. 238: 59-68. Neutrophils were harvested from rat whole blood and activated with phorbol 12-myristate 13-acetate (PMA). The activated neutrophils were then incubated and cultured in the presence of each of the test samples and the controls. The reduction of the WST-1 dye was measured to determine the products of superoxide. The control group was set at 100% activity (0% inhibition) and the inhibition of all samples were compared to this reference. Aspirin is a known anti-inflammatory compound and so was tested for a reference and exhibited 50.2% inhibition at a concentration of 400 μg/ml (16.5% inhibition was exhibited at a concentration of 100 μg/ml, and 48.12% inhibition was exhibited at a concentration of 200 μg/ml, showing a dose response effect).

The ethanol and water extracts (lipid rich and hydrophilic fractions) from each of the test samples were tested at 400 μg/ml for their anti-inflammatory activity based on the inhibition of superoxide by activated neutrophils. The yields of each extraction were used along with the activity of each to obtain an estimate for the total activity within each of the test samples for the two fractions. The results are shown in the tables below.

Ethanol Extracts—the Relative Contribution of the Lipid Fraction in Each of the Samples Tested for Anti-Inflammatory Activity

| | % of | | Weight (mg) of powder |
|---|---|---|---|
| | total | % Inhibition | required to achieve 100% |
| Sample No. | weight | (400 μg/ml) | inhibition of inflammation |
| 1 | 6.22 | 81.01 | 7.9 |
| 2 | 6.13 | 100 | Cannot be determined |
| 3 | 8.92 | 100 | Cannot be determined |
| 4 | 8.58 | 98.86 | 4.71 |
| 5 | 10.05 | 100 | Cannot be determined |

Water Extracts—the Relative Contribution of the Hydrophilic Fraction in Each of the Samples Tested for Anti-Inflammatory Activity

| | % of | | Weight (mg) of powder |
|---|---|---|---|
| | Total | % Inhibition | required to achieve 100% |
| Sample No. | Weight | (400 μg/ml) | inhibition of inflammation |
| 1 | 55.09 | 26.26 | 2.96 |
| 2 | 41.37 | 30.09 | 3.43 |
| 3 | 39.68 | 37.28 | 2.97 |
| 4 | 85.02 | 28.6 | 1.8 |
| 5 | 81.21 | 35.47 | 1.56 |

The results show that all of the samples showed some degree of anti-inflammatory activity, with the activity in the lipid fractions being higher than the activity in the hydrophilic fractions. It was however surprisingly discovered that there is anti-inflammatory activity in the hydrophilic fractions, so both the lipid and hydrophilic fractions of the mussel extracts contribute to the overall anti-inflammatory activity of the test samples. However, it is clear from the results that the test samples produced by the method of the invention give a much higher yield (almost 50% higher) of hydrophilic fraction than the test samples produced by conventional processing methods. FIG. 7 is a graph showing the results of this study in terms of the total bioactivity of the test samples (i.e. lipid and hydrophilic fractions combined) based on IC50 of aspirin equivalent (at 400 μg/ml). This graph shows that the test samples produced by the method of the invention show a much higher level of bioactivity than the test samples produced by conventional processing methods. The combination of the high yields of the hydrophilic fractions of the test samples produced by the method of the invention, with the good yields and high activity of the lipid fractions gives an overall increased bioactivity of the extracts as a whole. Very small dosages of the hydrophilic fraction of the test samples produced by the method of the invention are required in order to achieve 100% inhibition. Similarly, very small dosages of the extract as a whole would be required in order to achieve 100% inhibition. The results show that there is an increased yield of bioactive components produced by the method of the invention.

The results of this study are further supported by a subsequent study which was carried out on the same test samples (five ethanol extracts and five water extracts) to assess the DPPH scavenging activity of each of the test samples.

The antioxidant activity of each of the samples was tested using the DPPH scavenging method (i.e. by using the stable free radical 2,2-Diphenyl-1-(2,4,6-trinitrophenyl) hydrazyl as a substrate). Whilst the DPPH method is not a direct anti-inflammatory assay, antioxidant activity has been an indication of anti-inflammatory activity in many study cases. The DPPH solution was prepared in 0.1 mM ethanol and kept in the freezer in the dark before use. The positive control was ascorbic acid prepared as 0.1 mg/ml in a buffer containing citric acid and NaHPO₄ (pH 5). Equal amounts of sample solution and DPPH solution were added together, and the assay tube or plate was incubated for 30 minutes in the dark, followed by absorbance measurement at 517 nm on a spectrophotometer. In the blank control experiment of each sample, DPPH was replaced with ethanol. In the DPPH blank control experiment, the sample was replaced with the media (water or solvent) the sample was prepared in.

The scavenging activity (DPPH inhibition %) is calculated by percentage of the absorbance from the sample versus the DPPH only:

$$DPPH inhibition \% = [ 1 - \frac{( A sample - {A}_{sample blank} )}{( {A}_{DPPH} - {A}_{DPPH blank} )} ] \times 100$$

All samples were tested at a concentration of 10 mg/ml. The results showed that all samples had antioxidant activity (above 80% inhibition in all samples). The results are summarised in the tables below:

The IC₅₀ Values of DPPH Inhibition in Water Extracted Samples

| Sample | 1 | 2 | 3 | 4 | 5 |
|---|---|---|---|---|---|
| IC₅₀ (mg/ml) | 6.90 | 2.96 | 3.72 | 3.01 | 3.02 |

The IC₅₀ Values of DPPH Inhibition in Ethanol Extracted Samples

| Sample | 1 | 2 | 3 | 4 | 5 |
|---|---|---|---|---|---|
| IC₅₀ (mg/ml) | 8.12 | 6.34 | 7.73 | 4.05 | 4.50 |

These results are shown in FIG. 8 which is a graph showing the results of this study in terms of the total bioactivity of the test samples (i.e. lipid and aqueous or hydrophilic fractions combined) based on IC50 of Vitamin E equivalent (at 9.26 μg/ml). It is clear from the results that the samples produced by the method of the invention exhibit stronger antioxidant activity than the samples produced by conventional processing methods. Both the water extracts and the ethanol extracts contribute to the overall antioxidant activity.

### Example 2

Green-lipped mussels (Perna canaliculus) were processed using the same method as described in Example 1, except that a heat stabilisation step was carried out after the enzyme treatment step in order to denature the enzymes. The heat stabilisation step was carried out by applying a further steam injection into the treatment chamber to raise the internal temperature of the treatment chamber to a temperature of >80° C. for about 5-15 minutes. This example was carried out in order to determine whether or not the heat stabilisation step had any effect on the resulting bioactivity of the composition. A further bioactivity study to determine antioxidant activity (by way of DPPH scavenging activity) was conducted in respect of the composition produced in Example 2 in comparison to the composition produced in Example 1 and a composition produced by conventional processing methods (from the same batch of mussels). It was found that the heat stabilisation step had no significant effect on the bioactivity of the composition of Example 2. Results of the study showed very similar levels of antioxidant activity as the composition of Example 1 and higher antioxidant activity compared to the composition produced by conventional processing methods.

### Example 3

Green-lipped mussels (Perna canaliculus) were processed by adding 60 kg of live whole mussels to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The target substrate was protein and the enzyme treatment step involved application of 6% of an enzyme formulation (based on the total protein amount of between 3-4 kg) comprising a protease enzyme derived from Bacillus species (Bacillus amyloliquefaciens, commercially available as NEUTRASE). No further heating step was used as the internal temperature of the chamber was maintained at about 55-60° C. by the initial steam injection. The chamber was rotated for a period of about 40 minutes. The chamber was deactivated by activation of the exhaust which expelled the heat and pressure from the chamber. The contents of the chamber were then discharged onto a separating screen to remove any residual shells, shell fragments and any other large particles. The residual shells and shell fragments were very clean, both inside and out. The remaining liquid composition was filtered and stabilized.

The liquid composition produced in Example 3, together with two control samples produced by conventional mechanical processing methods were tested for anti-inflammatory activity using a Cyclooxygenase (COX, also called prostaglandin H synthase or PGHS) assay. Cyclooxygenase is a bifunctional enzyme exhibiting both COX and peroxidase activity. Recent research has established that there are two distinct isoforms of COX: COX-1 and COX-2. COX-1 is expressed in a variety of cell types and involved in normal cell biology. COX-2 is induced by mitogenic stimuli (LPS and cytokines) and is responsible for the biosynthesis of prostaglandins (PGs) under acute inflammatory conditions and therefore it is a target enzyme for the anti-inflammatory activity of nonsteroidal anti-inflammatory compounds. An ideal anti-inflammatory candidate should only possess COX-2 inhibition, not COX-1 inhibition.

COX-2 colorimetric inhibitor screening assay kits from Cayman Chemical Company (MI, USA) were used. A test sample of the liquid composition of Example 3 was prepared by extraction of the liquid composition with DMSO media as 100 mg/ml, then dilution of the sample in PBS to a concentration of 5 mg/ml. Comparative sample 1 was produced by manually opening green-lipped mussels, extracting the flesh and homogenising the flesh followed by extraction with DMSO media as 100 mg/ml, then dilution of the sample in PBS to a concentration of 5 mg/ml. Comparative sample 2 was produced by manually opening green-lipped mussels, extracting and incubating the flesh at 55° C. for 60 minutes then homogenising the flesh followed by extraction with DMSO media as 100 mg/ml, then dilution of the sample in PBS to a concentration of 5 mg/ml.

The results of the test are shown in FIG. 13. The results show that the extracts of the composition produced by the method of Example 3 had a very high level of COX-2 inhibition activity compared to comparative samples 1 and 2.

COX-2 inhibition activity is linked to anti-inflammatory activity and it is expected that due to the structure and properties of the compositions of the invention, they comprise a high yield of bioactive components with anti-inflammatory activity and improved bioavailability and will therefore be very effective in treating inflammation and associated conditions.

### Example 4

Green-lipped mussels (Perna canaliculus) were processed by adding 60 kg of live whole mussels to a sealable, pressurisable treatment chamber. The chamber was closed and subjected to a warming step with gentle agitation to achieve an optimum temperature of about 45-50° C. distributed inside the chamber. The process involved two enzyme treatment steps. The first step was carried out on the protein substrate using 6% of an enzyme formulation (based on the total protein amount of between 3-4 kg) comprising a protease enzyme derived from Bacillus species (commercially available as ALCALASE or ESP153 or ENZIDASE PTX6L) (alternatively a combination of all of these enzymes in various ratios making up a total concentration of about 6% could be used in the enzyme formulation). The internal temperature of the chamber was maintained at about 55-60° C. for a duration of 40 minutes. The chamber was deactivated by activation of the exhaust which expelled the heat and pressure from the chamber. The contents of the chamber were discharged and separated. The residual shells and shell fragments were very clean, both inside and out. The remaining liquid composition was filtered and the material remaining after filtration was added back into the treatment chamber and treated with a different enzyme formulation to act on the partially reduced protein substrate using 5% of an enzyme formulation (based on the total protein amount of between 3-4 kg) comprising a protease enzyme derived from Bacillus species (commercially available as Neutrase). The chamber was rotated for about 30 minutes at 55−60° C. Then it was deactivated and the contents of the chamber were collected. It was found that the second addition of enzyme formulation had improved the soluble protein yield in the liquid composition as well as significant particle size reduction.

### Example 5

Green-lipped mussels (Perna canaliculus) were processed according to the method of the invention. 60 kg of live whole mussels were added to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The process involved use of a mixed enzyme formulation selected to act on the protein substrate using 6% of an enzyme formulation (based on the total protein amount of between 3-4 kg) comprising a protease enzyme derived from Bacillus species (commercially available as ALCALASE or ESP153 or ENZIDASE PTX6L) combined with 5% (based on the total protein amount) of another enzyme derived from Aspergillus oryzae (commercially available as FLAVOURZYME or Lecitase® Ultra) at 25−55° C. No further heating was needed to maintain the reaction temperature. The treatment chamber was rotated for about 60 minutes. The chamber was deactivated. The contents of the chamber were then discharged onto a separating screen and the liquid composition was filtered through a 200 μm mesh filter. The liquid composition comprised an emulsion-like composition of suspended particles (in an aqueous medium) of uniform size and distribution. It was found that the combined enzyme formulation had reduced the particle size further and achieved a better tasting profile in the liquid composition.

### Example 6

Green-lipped mussels (Perna canaliculus) were processed according to the method of the invention. 60 kg of live whole mussels were added to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The process involved two enzyme treatment steps. The first step was carried out on the protein substrate using 6% of an enzyme formulation (based on the total protein amount of between 3-4 kg) comprising a protease enzyme derived from Bacillus species (commercially available as ESP153). The internal temperature of the chamber was maintained at about 55-60° C. The chamber was rotated for a period of about 40 minutes. The chamber was deactivated and the contents of the chamber were then discharged onto a separating screen. The liquid composition was filtered and the material remaining after filtration was added back into the treatment chamber and treated with a different enzyme formulation to act on the following target substrates: collagen, glycosaminoglycan and some complex carbohydrates and proteins using about 1% of an enzyme formulation of papaya (commercially available as PAPAIN 6000 L). An alternative enzyme formulation derived from Bacillus amyloliquefaciens (commercially available as ENZIDASE Neutral or NEUTRASE) could have been used if desired. Steam was injected to the chamber in order to raise the temperature to between 55-80° C., for a reaction time of about 30 minutes, before discharging the liquid composition. It was found that the second enzyme treatment step improved the soluble yield of the target substrates in the liquid composition.

### Example 7

Green-lipped mussels (Perna canaliculus) were processed by adding 60 kg of live whole mussels to a treatment chamber. The chamber was closed and a warming step employed to achieve an optimum temperature of about 45-50° C. The process involved two enzyme treatment steps. The first step was carried out on the protein substrate using 6% of an enzyme formulation (based on the total protein amount of between 3-4 kg) comprising a protease enzyme derived from Bacillus species (commercially available as ENZIDASE PTX6L). The internal temperature of the chamber was maintained at about 55-60° C. by steam injection. The chamber was rotated for a period of about 40 minutes. At the end of this time the chamber was deactivated by activation of the exhaust which expelled the heat and pressure from the chamber. The contents of the chamber were then discharged onto a separating screen to remove any residual shells, shell fragments and any other large particles. The liquid composition was filtered and the material remaining after filtration was added back into the treatment chamber and treated with an enzyme formulation comprising Trypsin in a concentration of about 2.5%, to act on the following target substrates: complex carbohydrates, lipids and proteins at a temperature of between 30-65° C. for about 120 minutes, before discharging the liquid composition. It was found that the second enzyme treatment step improved soluble yield of target substrates, that is, more lipids, free fatty acids, carbohydrates and protein/peptides were released from the complex matrix of the liquid emulsion in the soluble fraction.

### Example 8

Nine samples of green-lipped mussels (Perna canaliculus) from the same batch were prepared according to the method of the invention, using fresh post-mortem mussels, but different enzymes at different concentrations were used for processing each sample. The first three samples were processed by the addition of an enzyme formulation comprising ESP 153 (Connell Bros, Australia. Batch no. 7947) at three different concentrations, being 0.5%, 1% and 2% of total protein amount based on 15% weight of whole fresh mussel starting material. The next three samples were processed by the addition of an enzyme formulation comprising Neutrase 0.8 L (Novozyme, Denmark) at concentrations of 1.5%, 3% and 6% respectively. The final three samples were processed by the addition of an enzyme formulation comprising papain (Connell Bros, batch No. 8849) added in amounts of 10 mg, 20 mg, and 30 mg, respectively. All hydrolysis was carried out at 55° C. with gentle agitation. The degree of hydrolysis was evaluated after durations of 20 minutes, 50 minutes and 90 minutes respectively, in order to evaluate the effects of enzyme concentration and enzyme treatment time on the degree of hydrolysis.

A laboratory control sample comprising fresh homogenised mussel meat from the same batch of mussels placed in a flask at 55° C. with gentle agitation for the same 90 minute duration (with no enzyme added) was also tested.

The results showed that at all concentrations of enzyme added, in the first 20 minutes, the rate of hydrolysis was at its fastest, then slowed to a plateau at near 90 minutes. In all cases the degree of hydrolysis increased slightly as the concentration of enzyme increased. The use of 1% ESP153 achieved a similar degree of hydrolysis as that with 3% Neutrase, and the use of 2% ESP153 achieved a similar degree of hydrolysis as that with 6% Neutrase. The use of 30 mg papain achieved a similar degree of hydrolysis as 0.5% ESP153 and 1.5% Neutrase respectively, indicating that a higher concentration of papain is more effective. The results showed that different types and concentrations of enzymes can be used in the enzyme formulations of the invention and still achieve effective separation of biological material from the shells of the mussels and liquefication of the biological material within 20 minutes.

It was also noticed that a small degree of hydrolysis occurred in the control sample, confirming the existence of endogenous proteases in the mussel starting material and the release of these enzymes following homogenisation, thereby triggering autolysis which would cause damage and degradation of bioactive components in the mussel material.

COX-2 Inhibition Activity

Three of the above samples were evaluated for their COX-2 inhibition activity, using the same method as described in Example 3. One sample was chosen randomly from each trio of samples so that one sample produced using each of the enzyme formulations was tested for COX-2 inhibition activity. Each sample was prepared at the end of the 90 minute enzyme treatment process by extraction with DMSO as in Example 3. Sample 1 was from the batch treated with 2% ESP153. Sample 2 was from the batch treated with 1.5% Neutrase. Sample 3 was from the batch treated with 0.08% papain. The control was also tested. Each sample was tested at a dosage rate of 5 mg/ml. The results are shown in FIG. 14. The results showed that all of the samples achieved a good level of COX-2 inhibition activity compared to the control. Given that the degree of hydrolysis at the end of 90 minutes was only slightly higher than that at 20 minutes, it is likely that the same results would have been achieved had the samples been tested for COX-2 inhibition activity after an enzyme treatment step of only 20 minutes.

### Example 9

Green-lipped mussels (Perna canaliculus) were processed by adding 60 kg of live whole mussels to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The process involved a single enzyme treatment step carried out on the protein substrate using a combined enzyme formulation (based on the total protein amount of between 3-4 kg) comprising two enzymes derived from Bacillus species namely ESP153 and NEUTRASE and one cysteine protease enzyme, namely papain. The enzyme formulation comprised enzymes in the amounts of 2-3% ESP153 and 3-5% NEUTRASE and 0.2-0.3% PAPAIN. No further heating step was used as the internal temperature of the chamber was maintained at about 55-60° C. by the initial steam injection. The duration of the enzyme treatment step was 60 minutes. After separation, the residual shells and shell fragments were very clean, both inside and out. The liquid composition was filtered and stabilized. The resultant composition was stable with a consistent particle size and structure and a high soluble yield of target substrates.

### Example 10

Green-lipped mussels (Perna canaliculus) were processed according to the method of Example 9, however two enzyme treatment steps were carried out. The first step was carried out on the protein substrate using an enzyme formulation (based on the total protein amount of between 3-4 kg) comprising a proteolytic enzyme derived from Bacillus species (commercially available as NEUTRASE) in an amount of 2% for 30 minutes. No further heating step was used as the internal temperature of the chamber was maintained at about 55-60° C. by the initial steam injection. After 30 minutes another enzyme formulation comprising a mixture of three other enzymes: 2% ESP153, 60 mg of papain, and 4% of Lecitase Ultra (Novozyme) was added to the treatment chamber. Processing was continued for a further 60 minutes. The chamber was deactivated and the liquid composition was collected. It was found that the second enzyme treatment step improved the soluble yield of the target substrates in the liquid composition. The resultant composition was stable with a consistent particle size and structure.

### Example 11

Blue mussels (Mytilus edulis) were processed by the addition of 60 kg of live whole mussels to a treatment chamber. The chamber was closed and a warming step in the form of a flash steam injection employed to achieve an optimum temperature of about 45-50° C. together with gentle rotation. The enzyme treatment comprised application of 6% of an enzyme formulation (based on the total protein amount of between 3-4 kg) comprising a proteolytic enzyme derived from Bacillus species (commercially available as ALCALASE). Alternative enzymes such as ESP153 or ENZIDASE PTX6L or NEUTRASE could also have been used. The internal temperature of the chamber was maintained at about 55-60° C. by steam injection. The chamber was rotated for a period of about 30 minutes. At the end of this time the chamber was deactivated by activation of the exhaust which expelled the heat and pressure from the chamber. The contents of the chamber were then discharged onto a separating screen to remove any residual shells, shell fragments and any other large particles. The remaining liquid composition was filtered and stabilised. It was observed that the structure and stability of the composition was substantially similar to those prepared with green-lipped mussels, indicating that the method of the invention is effective regardless of mussel species.

### Example 12

New Zealand Cockles (Austrovenus stutchburyi) were processed according to the method of the invention. 60 kg of whole live cockles were added to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the cockles. 6% of an enzyme formulation comprising a protease enzyme derived from Bacillus species was applied. The internal temperature of the chamber was maintained at about 55-60° C. by the initial steam injection. The chamber was rotated for a period of about 20 minutes. The chamber was deactivated and the contents of the chamber were discharged onto a separating screen. The liquid composition was filtered and stabilised. It was observed that the structure and stability of the composition was substantially similar to those prepared with mussels, indicating that the method of the invention is effective regardless of the bivalve species used.

### Advantages

The method and compositions of the invention have the following potentially realisable advantages:
a) Increased yield of bioactive components in the resulting liquid and dried compositions;
b) Utilisation of substantially all of the useful biological material present in or on the shellfish starting material meaning very little waste or yield loss;
c) Higher quantitative yields of both liquid and dried compositions;
d) Quicker process (whole live shellfish can be processed in less than two hours, but generally in less than 40 minutes which is a huge advance on prior art enzyme hydrolysis methods which take many hours);
e) Simplified process (significantly less steps involved and less equipment involved than in prior art processes);
f) Process does not require the use of mechanical techniques or high temperatures that will denature or destroy bioactive components;
g) More cost effective and efficient process due to less equipment, less time and less steps required;
h) Many possible end products including intermediate products and finished products;
i) Minimal treatment time and processing means chances of contamination, oxidation, loss, damage or degradation of bioactive components of the shellfish is greatly reduced;
j) Characteristics of resulting compositions allow for multiple drying options to be used, and for multiple fractionation, separation and extraction processes to be used to make further extracts and/or product formats;
k) The process yields highly soluble extracts which are more easily formulated, providing more options for further processing and uses;
l) No anti-oxidants are required to be added during the process of the invention;
m) The compositions have improved sensory attributes including smell, taste and flavour profiles and the improved sensory attributes make the compositions suitable for use in many different applications;
n) The compositions have unique food matrices in the form of emulsion-like compositions and/or self-emulsifying compositions which have high solubility in aqueous mediums, and putated high bioavailability due to an increased ability to be absorbed into the body. The highly desired lipid or lipophilic bioactive components are naturally protected or encapsulated in the compositions thereby increasing the bioavailability and efficacy of these desired bioactive compounds.
o) The compositions are naturally stable and completely natural since they form natural emulsion-like compositions which do not require the addition of any surfactants, co-surfactants or other emulsifying agents or additives to stabilise the compositions.

### Variations

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

While the examples show methods carried out using certain enzymes and/or enzyme combinations, these show some preferred enzyme formulations only. It is envisaged that most commercially available enzymes would be effective in the method of the invention, and the actual selection of enzymes themselves is determined by reference to a number of factors such as the optimum processing temperature and pH of each enzyme, the time it generally takes to obtain a sufficient degree of hydrolysis with each enzyme type, the respective costs and general accessibility of different types of enzymes. In addition, the species of shellfish and target substrates of the shellfish should be considered as well as the desired end-products and specifications.

In the examples, mussels are primarily used as the starting shellfish material. However, it is expected that the processing method will work in a similar manner in respect of other shellfish species and trials are about to be conducted to show this. Initial studies show that similarly structured and therefore advantageous compositions are able to be achieved with different mussel species and different bivalve species. It is possible that different enzymes may need to be used with different shellfish species, depending on their biological make-up. The bioactive components present in the end-products produced from different shellfish species will differ depending on the species, but it is envisaged that a large proportion of the potentially beneficial bioactive components that are present in the shellfish starting material will be recovered and have high bioavailability in the compositions of the invention. Other potentially beneficial bioactive components may also be released through the method of the invention.

It will also be understood that where a product, method or process as herein described or claimed is sold incomplete, as individual components or as a “kit of parts”, or is carried out as individual or separate steps, that such exploitation will fall within the ambit of the invention.

For purposes of the description hereinafter, the terms “upper”, “lower”, “right”, “left”, “vertical”, “horizontal”, “top”, “bottom”, “lateral”, “longitudinal”, “side”, “front”, “rear” and derivatives thereof shall relate to the invention as it is oriented in the drawing figures. However it is to be understood that the invention may assume various alternative variations, except where expressly specified to the contrary. It is also to be understood that the specific devices illustrated in the attached drawings, and described in the following specification are simply exemplary embodiments of the invention. Hence specific dimensions and other physical characteristics related to the embodiments disclosed herein are not to be considered as limiting.

1.-51. (canceled)

52. A large-scale process of preparing a composition from whole fresh shellfish of the class Bivalvia, wherein the process includes the steps of:
(a) gapping or at least partially opening or penetrating the shells of the bivalves;
(b) applying an enzyme formulation comprising one or more enzymes to the bivalves;
(c) leaving the bivalves in contact with the enzyme formulation until the flesh and other biological materials are substantially liquefied and separated from the shells of the bivalves;
(d) separating the shells from the liquefied composition.

53. The process of claim 52 wherein the shellfish are alive at or up to the point of step (a) or step (b).

54. The process of claim 52 wherein step (a) comprises application of heat to the bivalves for a short period of time to raise the internal temperature of the bivalves to about 35-55° C.

55. The process of claim 54 wherein the heat is applied by flash steam injection or infusion.

56. The process of claim 52 wherein the duration of step (c) is less than 90 minutes.

57. The process of claim 52 wherein the duration of step (c) is less than 45 minutes.

58. The process of claim 52 wherein steps (b)-(c) are carried out at a controlled temperature in the range of about 20-60° C.

59. The process of claim 52 wherein the enzyme formulation comprises at least one enzyme selected from the group comprising cysteine proteases and collagenases, for acting on the abductor muscles of the bivalves in order to facilitate full opening of the shells.

60. The process of claim 52 wherein the enzyme formulation comprises one or more enzymes selected from the group comprising Bacillus amyloliquefaciens, Bacillus licheniformis, Bacillus subtilis, Aspergillus niger, Aspergillus oryzae; cysteine proteases; serine proteases; trypsin; collagenases; carbohydrases; phosphatases; lipases; phospholipases; catalases; and combinations thereof.

61. The process of claim 52 wherein the process includes a step of stabilising the liquified composition before or after step (d).

62. The process of claim 52 wherein steps (a)-(c) are carried out in a single closed temperature-controlled treatment vessel or chamber.

63. The process of claim 52 wherein the process includes at least one filtering step after step (d).

64. The process of claim 63, wherein the filtering step comprises filtering the liquefied composition to a particle size of less than 200 μm.

65. The process of claim 52 wherein the process further includes a drying step to obtain a dried shellfish composition.

66. The process of claim 52 wherein the process further includes at least one fractionation step to separate desired components of the resulting shellfish composition.

67. A liquid shellfish composition produced by the process of claim 52.

68. A dried shellfish composition produced by the process of claim 65.

69. A lipid shellfish extract produced by the process of claim 66.

70. A non-lipid shellfish extract produced by the method of claim 66.

71. A product comprising a dried shellfish composition as claimed in claim 68.
